(19) United States
(12) Patent Application Publication
(10) Pub. No.: US 2022/0068501 A1
(43) Pub. Date: Mar. 3, 2022

(54) SYSTEM AND METHOD FOR TRACKING SENSITIVE MEDICATIONS

(71) Applicant: Autonomous Healthcare, Inc., Hoboken, NJ (US)

(72) Inventors: Behnood Gholami, Hoboken, NJ (US); Amir Avnit, New Haven, CT (US); Deepak Khemraj, South Ozone Park, NY (US)

(21) Appl. No.: 17/463,851

(22) Filed: Sep. 1, 2021

Related U.S. Application Data
(60) Provisional application No. 63/073,053, filed on Sep. 1, 2020.

(51) Int. Cl. G16H 70/40; G16H 20/10; G06F 21/32

(52) U.S. Cl. CPC G16H 70/40; G06N 20/00; G06F 21/32; G16H 20/10

(57) ABSTRACT

The present disclosure describes a system, apparatus, and method involving immutable databases such as blockchains to track and document transportation and administration of sensitive medications and identify potential cases of drug diversion. The disclosed invention uses a transaction recording module to scan unique tags for sensitive medications packs during the transport process. The invention also comprises an image and video capture module, an immutable database, a computation engine, a graphical user interface, and a notification module.

### CROSS-REFERENCE TO RELATED APPLICATIONS

This application relies on the disclosure of and claims priority to and the benefit of the filing date of U.S. Provisional Application No. 63/073,053, filed Sep. 1, 2020, the disclosure of which is hereby incorporated by reference herein in its entirety.

### GOVERNMENT RIGHTS

The invention was made with government support under 1 R43 DA051084-01 by the National Institutes of Health (NIH). The government has certain rights in the invention.

### BACKGROUND

Tracking the chain of custody and the use of sensitive medication is important to address challenges associated with supply chain and logistics of the manufacturing and distribution of medications and to follow governmental guidelines related to the use of medications. One form of sensitive medication is a controlled substance, where its use and possession are regulated by laws set by governments. Within this context, tracking of controlled substances is important to prevent drug diversion. A potential use of the invention is for the tracking of medications that are expensive, subject to regulations due to their nature (such as controlled substances), in high demand, or need to be authenticated to avoid counterfeit medications.

Drug diversion is defined as the transfer of any legally prescribed sensitive medication to a person or channel for any illicit use. Drug diversion is a multi-faceted problem involving many components, ranging from lack of prescription-provider training to failure to implement hardware and software systems to manage physical access, and lack of auditable data related to access to sensitive medications, including controlled substances. However, despite recent improvements in controlling and monitoring access to sensitive medications, the process of identifying drug diversion and ensuring compliance is still complicated, generally manual and ad hoc, and time consuming.

The disclosed invention involves an asset tracking or medication tracking system and apparatus where sensitive medication packs are tracked along the transportation chain starting from where the sensitive medication is stored to the point of administration to the patient or its waste and deliberate destruction end point (in situations where it is not administered to a patient). In some embodiments, the disclosed invention is used in a hospital or other facility that directly supports the identification of the need for a sensitive medication, the ordering of such medication for a patient, and optionally, the administration thereof. In such systems, the asset or medication physical transportation chain starts with the pharmacy vault where the controlled medication is logged and securely stored and ends with administration of the medication to the patient, the logging of that event, and the use of the sensitive medication as a result of the administration event within the system. In other embodiments, the disclosed invention is used to track medications throughout the supply chain process starting from the manufacturer, though distributors, and ending in the hospital, pharmacy, or other medical facility at the retail level. In such a system, the asset or medication physical transportation chain starts with its manufacturing—where sensitive medication is packaged and logged and securely stored for transportation to distributor—and ends in the delivery of such packages by distributors to the final destination such as a hospital or other facility that directly supports the identification of the patient's need for a sensitive medication, the ordering of such medication for a patient, and optionally, the administration thereof to them.

An embodiment of the disclosed invention involves a mobile application for logging events related to the transportation, administration, or waste of sensitive medication. In some embodiments, a smart phone or tablet is used for both logging of such events and capturing image and/or video data related thereto. In some embodiments, a web-based dashboard is used to monitor and visually represent, either in real-time or at a predetermined time period or based on a triggering event, the movement of medications and generate reports and identify outliers and anomalies in the process.

Additionally, the invention provides a method for recording transactions on a database including but not limited to an immutable database, as described in greater detail below. The invention also provides a method to detect potential fraudulent activities based on data recorded in the database.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the invention provides a system to track sensitive medication packs, comprising:
an immutable database configured to receive transaction data from at least one transaction recording module;
at least one transaction recording module comprising computer-executable instructions in operable communication with one or more computer processors, the at least one transaction recording module configured for and in operable communication with the immutable database for transmitting transaction data, wherein the at least one transaction recording module is configured to:
   a) verify the user identity using a biometric verification process and/or verifying a username and password combination with a previously recorded username and password for that user recorded in the immutable database;
   b) scan identification tags on sensitive medication packs;
   c) transmit transaction data to the immutable database; and
   d) receive a confirmation from the immutable database that transaction data was successfully recorded;
an image and/or video capture module for and in operable communication with the immutable database for assigning a unique identification number to a recorded image of the sensitive medication packs and/or a recorded video of the sensitive medication administration process and transmitting the recorded image and/or recorded video and the unique identification number to the immutable database; and
a computation engine module comprising computer-executable instructions in operable communication with one or more computer processors, the computation engine module configured for and in operable communication with the immutable database for:
   a) retrieving transaction data, the recorded image of the sensitive medication packs and/or the recorded video of the sensitive medication administration process from the immutable database;
   b) generating an audit trail associated with the scanned tag;
   c) retrieving a recorded image of the sensitive medication and/or recorded video of the sensitive medication administration process for audit purposes; and
   d) detecting a red flag related to drug diversion.

In additional embodiments, the apparatus of the present invention comprises the hardware, software, immutable database, at least one transaction recording module, and at least computation engine module described herein.

In some embodiments, the invention provides a method to track sensitive medication packs, comprising:
transmission of image and/or video capture information from at least one transaction recording module comprising computer-executable instructions in operable communication with at least one computer processor to an immutable database; wherein said image and/or video capture information contains a unique identification number linked to a recorded image of a sensitive medication pack or a recorded video of the sensitive medication administration process;
receipt of transaction data from said at least one transaction recording module; and
storage of said transaction data in an immutable database.
   Additionally, in some embodiments, the method includes transmission of additional information from the transaction recording module, through use of a transaction recording application configured for and in operable communication with the immutable database for transmitting transaction data, wherein the at least one transaction recording module takes the steps of:
a) verifying the user identity using a biometric verification process and/or verifying a username and password combination with a previously recorded username and password for that user recorded in the immutable database;
b) scanning identification tags on sensitive medication packs; and
c) transmitting this additional transaction data to the immutable database.
   Upon receipt and verification of the transmitted information, the immutable database will generate a confirmation that the transaction data was successfully recorded.
   Further, the method includes the step of communication between a computation engine module comprising computer-executable instructions in operable communication with one or more computer processors and an immutable database, wherein the computation engine module is configured for and in operable communication with the immutable database for:
a) retrieving transaction data, the recorded image of the sensitive medication packs and/or the recorded video of the sensitive medication administration process from the immutable database;
b) generating an audit trail associated with the scanned tag;
c) retrieving a recorded image of the sensitive medication and/or recorded video of the sensitive medication administration process for audit purposes; and
d) detecting a red flag related to drug diversion.

### BRIEF DESCRIPTION OF THE FIGURES

FIG. 1 illustrates the overall apparatus of the sensitive medication tracking system.

FIG. 2 illustrates an embodiment of the transaction recording graphical user interface related to retrieved drugs from a kit as part of a mobile application for a smartphone.

FIG. 3 illustrates an embodiment of the transaction recording graphical user interface related to administration, waste, return to the medical kit, transfer to a second person (e.g., a surgeon) or return to inventory of a specific drug (e.g., morphine sulphate) as part of a mobile application for a smartphone.

FIG. 4 illustrates an example witness waste barcode in the form of a “waste request” QR code, which is displayed on a transaction recording module implemented on as a mobile application on a smartphone.

FIG. 5 illustrates an embodiment of the computation engine graphical user interface related to current inventory at different locations.

FIG. 6 illustrates an embodiment of the computation engine graphical user interface related to displaying the current status of medical kits.

FIG. 7 illustrates an embodiment of the computation engine graphical user interface related to displaying transactions associated with a user “Deepak PHARMA”.

FIGS. 8A-C illustrate examples of state diagrams of Markov chains corresponding to different roles, used within the simulation. A) Pharmacist/Technician; B) Anesthetist/CRNA; C) Nurse.

FIG. 9 illustrates an example single-shift timeline, indicating each action logged for a user with an anesthetist role.

FIG. 10 illustrates anomaly score contour of an isolation forest model for synthetic training and test datasets generated by the simulation, and an artificial dataset of outliers.

FIG. 11 illustrates steps that the transaction recording module takes as part of recording transaction data and transmitting the data to the immutable database.

FIG. 12 illustrates the steps that the immutable database takes as part of recording transaction data and receiving such data from at least one transaction recording module.

FIG. 13 illustrates an embodiment of the overall workflow associated with using the disclosed invention for tracking sensitive medication in a hospital or healthcare facility.

FIG. 14 illustrates the steps that transaction recording module takes to approve a kit prepared by a pharmacist or a pharmacy technician or nurse or a person with a similar role.

### DETAILED DESCRIPTION

The invention disclosed herein describes a technology to track and document transportation, storage, wasting, and administration of sensitive medications, and identifying potential cases of drug diversion within a healthcare setting or throughout the supply chain environment.

In this disclosure, sensitive medication is defined as a drug or chemical such that its movement, use, custody, and disposal has to be accurately recorded. In some embodiments, sensitive medication is a controlled substance (a drug or chemical for which a government regulates its manufacture, possession, or use). In some embodiments, a drug with a high price or in high demand is a sensitive medication. In some embodiments, a drug, that needs to be authenticated to ensure that it is the authentic product of the approved source, and not a counterfeit product, is a sensitive medication. In this disclosure, “drug” means any drug, chemical, medication, vaccine, or compound that has a pharmacological effect on the human body, whether it is regulated by a governmental body, or not. In some embodiments, certain medications and treatments that are not regulated by the government, such as CBD-based and herbal treatments, are also considered a sensitive medication.

One component of the invention involves registering various transactions, records, and logs in certain databases. Example databases that can be utilized in the invention include SQL and NoSQL databases, and immutable databases including those based on the blockchain technology. The term immutable database is used to represent any database technology that has safeguards for tampering with stored data to generate an audit trail that cannot be altered, once recorded. Examples of immutable databases include blockchain protocol based distributed ledgers and Amazon Quantum Ledger. In some embodiments, the disclosed invention can be used in healthcare facilities, hospitals, rehabilitation clinics, long-term care and assisted care facilities, ambulatory surgical centers, among other environments. In such situations, a private or hybrid blockchain deployment model may be used. In other embodiments, a permissioned blockchain can be used as an immutable database. A permission blockchain ensures that the data remains private and is not shared outside the appropriate entities or organizations and user stakeholder groups participating in the relevant healthcare system. Immutable database concepts are discussed in greater detail in the literature including discussions provided in Narayanan, Arvind, et al. “Bitcoin and cryptocurrency technologies: a comprehensive introduction.” Princeton University Press, 2016.

Any algorithm described herein can be embodied in software or a set of computer-executable instructions capable of being run on a computing device or devices. The computing device or devices can include one or more processors (CPU) and a computer memory. The computer memory can be or include a non-transitory computer storage media such as RAM which stores the set of computer-executable (also known herein as computer readable) instructions (software) for instructing the processor(s) to carry out any of the algorithms, methods, or routines described in this disclosure. As used in the context of this disclosure, a non-transitory computer-readable medium (or media) can include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The computer-readable instructions can be programmed in any suitable programming language, including JavaScript, C, C#, C++, Java, Python, Perl, Ruby, Swift, Visual Basic, and Objective C. Embodiments of the invention also include a non-transitory computer readable storage medium having any of the computer-executable instructions described herein.

A skilled artisan will further appreciate, in light of this disclosure, how the invention can be implemented, in addition to software and hardware, using one or more firmware options. As such, embodiments of the invention can be implemented in a system which includes any combination of software, hardware, and firmware technologies. In the context of this specification, the term “firmware” can include any software programmed onto the computing device that interacts with, takes instruction from, or provides instructions to the computer hardware, such as a device's nonvolatile memory. Thus, systems of the invention can also include, alternatively or in addition to the computer-executable instructions, various firmware modules configured to perform the algorithms that support the invention.

The computing device or devices can include a mainframe computer, web server, database server, desktop computer, laptop, tablet, netbook, notebook, personal digital assistant (PDA), gaming console, e-reader, smartphone, or smartwatch, which may include a processor, memory, hard drive, graphics processing unit (GPU), and input/output devices such as a display, keyboard, and mouse or trackpad (depending on the device). Embodiments can also provide a graphical user interface (GUI) made available on one or more client computers. The GUI can allow a user on a client computer to gain remote access to the results of the algorithm's activities.

Additional embodiments of the invention can include a networked computer system for carrying out one or more methods of the invention. The computer system can include one or more computing devices which can include a processor for executing computer-executable instructions, one or more databases, a user interface, and a set of instructions (e.g., software) for carrying out one or more methods of the invention. According to other embodiments, the computing device or devices can be connected to a network through any suitable network protocol such as IP, TCP/IP, UDP, or ICMP, such as in a client-server configuration and one or more networked database servers. The network can use any suitable network protocol and can be any suitable wired or wireless network including any local area network, wide area network, Internet network, telecommunications network, Wi-Fi enabled network, or Bluetooth enabled network.

In the context of the present invention, the term sensitive medication packs refers to one or more vials, ampules, syringes, prefilled syringes, packs, boxes, blister packs, and/or bottles (and/or other mediums for transfer of pills, tablets, capsules, intravenous injectable drugs, or any other form that sensitive medications are delivered for clinical use) of sensitive medications. The disclosed invention uses a transaction recording module to scan tags. In this disclosure, the term “scan tags” or “scan identification tags” or other variations thereof means scanning or reading of barcodes (linear or 2D barcodes such as QR code, datamatrix, databar, among others) printed on sensitive medications packs (or added by a secondary label) during the transport, delivery, administration, and waste process. In some embodiments, tags are generated by a computer application and printed as a linear or 2D barcode or a QR code on a paper-based sticker or directly printed on packaging material. In some embodiments, such tags may be RFID tags, nearfield communication tags, or other electromagnetic or optical identification tags.

The tags on sensitive medication packs may be unique (i.e., each unique encoded number can be specifically associated with one single pack) or non-unique (i.e., each encoded number may be associated with a batch of products like a formal manufacturing lot or run). In some embodiments, the tag is not unique and is used to encode the drug contents using the national drug code (NDC) by the Food and Drug Administration (FDA), for example. In this disclosure, scanning the tags means automated processing of an image or series of images to read barcodes and/or using computer vision algorithms including optical character recognition an neural network approaches for character recognition and other methods to read alphanumeric characters using image data to extract written information on the sensitive medication packs such as NDC, expiration date, serial number, and lot number, among others.

Sensitive Medication Apparatus

The overall apparatus comprises one or more transaction recording modules, a database module, one or more image and video processing modules, and a computation engine module. The computation engine module is an optional component of the system and in some embodiments only one or more transaction recording modules, a database module, one or more image and video processing modules exist. The transaction recording module captures various information related to movement and chain of custody information for sensitive medication packs. In some embodiments, the image and video processing module is a subcomponent of the transaction recording module. All transactions are recorded in a local recording log in the transaction recording module and communicated with the database. The database module records and archives all data captured by the transaction recording module. In some embodiments, the database module is hosted on a local or cloud server. The transaction recording module has a transaction module graphical user interface subcomponent. At least one or more interactions with the user to gather sensitive medication pack transaction data including scanning of identification tags are through this transaction module graphical user interface subcomponent. A computation engine module reviews and analyzes all transactions recorded in the database and images and/or videos to find any anomalies, outliers, policy violations, and/or any indication of suspicious behavior as related to a sensitive medication such as drug diversion. The computation engine module also includes a GUI subcomponent to communicate the results of the computation engine to the user. It also has a notification module subcomponent to identify certain transactions that require the attention of a user and to transmit notifications related to any identified time-sensitive transactions or anomalies to the user through a smartphone or email or other means. Example notifications include the request for a pharmacist to approve a kit, a request to witness a drug waste and identification of suspected policy or legal violations to be reviewed by a person with an administrative role, among others.

A transaction is defined as an event together with all related data (such as time and date, location, people involved, drug data such as NDC, expiration date, lot number, or serial number, and/or any unique or non-unique identification tags) pertaining to that event that involves movement, use, possession, disposal, retrieval, exchange with a second person, and other related actions for sensitive medications.

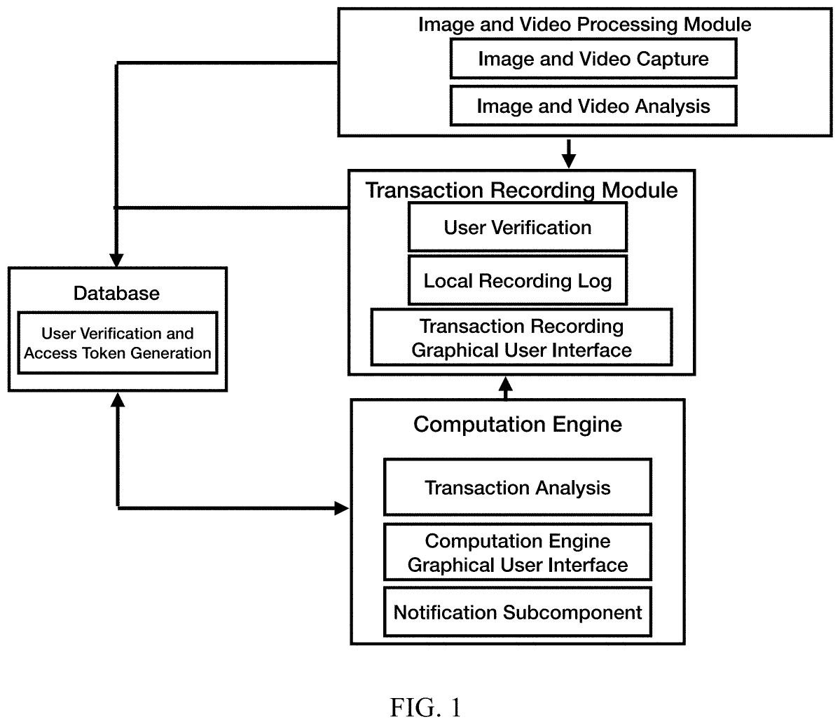

FIG. 1 illustrates the overall apparatus of the sensitive medication tracking system, the constituent modules, and related subcomponents.

Transaction Recording Module

A transaction recording module is used to record various transaction information related to sensitive medication packs. These transactions are described in detail below. The transaction recording module is composed of a transaction recording GUI to support interactions with the user. The transaction recording module also has access to image and video through the image and video processing module. In some embodiments, a smartphone is a transaction recording module, where a GUI is designed as part of a mobile application which uses the smartphone camera for image and video capture activities. The transaction recording module has a local recording log to record transaction data and communicate with the database module.

The transaction recording module has a user verification subcomponent responsible for communication with the user verification and access token generation subcomponent of the database module to verify the identity of the user. The transaction recording module can verify the identity of the user using i) a biometric verification process such as fingerprint scanning or facial feature scanning which is built-in to the host device or using a separate device; and/or ii) verifying a username and password combination with a previously recorded username and password for that user recorded in a remote database, that is, a database hosted remotely (e.g., on the cloud or on a local server). In some embodiments, the transaction recording module sends the username and password or the fingerprint scan or facial feature scan characteristics to the database for verification against existing authorized users. In some embodiments, the transaction recording module performs verification once and relies on the module's built-in fingerprint and facial feature scanning technologies to confirm the identity of the user without communication with the remote database.

The transaction recording module can transmit transaction data comprising one or more of the verified user identities, scanned identification tag of the sensitive medication, identification tag of the patient, current location of the scanned sensitive medication packs and current time and date, to the remote database (a standard relational database, SQL or NoSQL database, or immutable database among others) for recording. The transaction recording module can receive a confirmation from the database that the transmitted transaction data was successfully recorded. Transmission of the information can be performed over a network or through other communication means such as ethernet, Wi-Fi or Bluetooth. In some embodiments, a smartphone application is a transaction recording application.

In some embodiments, the user or users can digitally sign medication transfers between staff using digital transfer certificates as a replacement for the prevalent paper-based transfer systems. The digital transfer certificate is an electronic transaction that records the transfer of custody of a sensitive medication pack from the first person to a second person.

In some embodiments, more than one transaction recording module is used. Each transaction recording module communicates with the database to transmit transaction data and receive a confirmation that such transaction data was recorded. Transaction data comprises the verified user identity performing the transaction recording, user identity of other persons involved in the transaction, scanned identification tag of the sensitive medication pack, current location of the scanned sensitive medication packs and current time and date, and identification tag of the patient.

In some embodiments, the recording module also incorporates digital signatures when sensitive medications physically change hands between different people.

Transactions related to pharmacy: In some embodiments, the transaction recording module records various transactions in operations related to pharmacy such as receipt of inventory, storage, and preparation of medication kits or totes or other containers (all referred herein as kits) to be used for clinicians during patient care. The pharmacist or pharmacy technician can use the transaction recording module to refill kits (where kits may have a unique assigned tag or barcode). In this process, the transaction recording module records the kit number (by reading and/or scanning the kit tag). Then all sensitive medication packs that are added or removed from the kit are recorded through one or more transactions. In some embodiments, the transaction recording module verifies the NDC of the kit being added through image analysis of the sensitive medication pack provided by the image and video processing module. In some embodiments, a unique ID encoded in a tag and available on the sensitive medication pack is recorded. The transaction recording module is used to record all changes made to kits and provides details of all contents for a pharmacist to review and approve the kit prior to providing the kit to a clinical user for administration to a patient.

In some embodiments, the transaction recording module also is used to record receipt of new medications to be added to the inventory or removed from inventory. The process includes adding the details of such transactions such as the drug names, NDC, and quantity. In some embodiments, transaction details are automatically extracted by the image and video processing module through optical character recognition (OCR) or other computer vision methods from a hard copy printout. In some embodiments, transaction details are entered manually using a transaction recording graphical user interface.

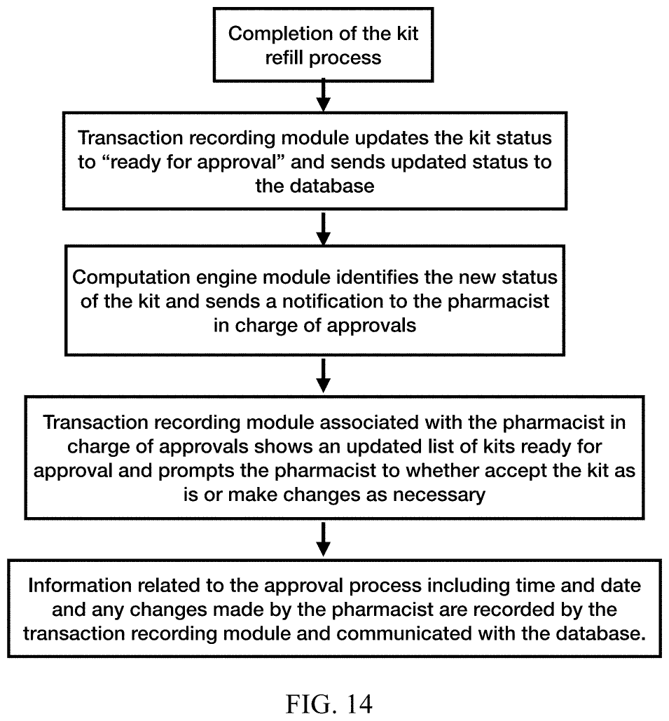

In some embodiments, the transaction recording module is used to approve a kit prepared by a pharmacist or a pharmacy technician or nurse or a person with a similar role. Once the person preparing the kit confirms the completion of the kit preparation/refill and reviews its contents, the transaction recording module updates the status of the kit to “ready for approval” and communicates this updated status with the remote database. The computation engine, which is responsible for continuous review of the database information, detects the updated change in status and sends a notification to the transaction recording module associated with the pharmacist in charge of the approval process. The transaction recording module associated with the pharmacist in charge of the approval process provides an updated list of kits ready for approval and prompts the pharmacist to accept the kit as is or make changes as necessary. Information related to the approval process including time and date and any changes made by the pharmacist are recorded by the transaction recording module and communicated with the database. FIG. 14 illustrates the steps that transaction recording module takes to approve a kit prepared by a pharmacist or a pharmacy technician or nurse or a person with a similar role.

Transactions related to patient care process: In some embodiments, the transaction recording module records transactions related to sensitive medication packs during the patient care process. In various clinical scenarios such as pre-operative, intra-operative, post-operative procedures, and for patient care in various settings such as standard care and critical care, sensitive medication packs need to be handled by clinicians (e.g., physicians, nurses, anesthetists, etc.). In order to have an auditable trail of handling of sensitive medications (retrieval from a medical kit, cart, or storage, administration, handing off to other personnel, wasting the drug, or return to the medical kit, cart, or storage), a transaction recording module is used. In some embodiments, the transaction recording module records time and date of the transactions, location (through GPS coordinates), the personnel involved, and the nature of the transaction and any other comments or notes the user enters into the system.

In some embodiments, the transaction recording module records transaction as related to a medical kit (or tote or other container) used by a clinician such as an anesthetist. In some embodiments, transactions include retrieving a medication from the kit (with an assigned identification tag for the kit) prior to use. In this process, the transaction recording module records the kit number (by reading and/or scanning the kit tag). Then all sensitive medication packs that are retrieved from a kit prior to clinical use are recorded through one or more transactions. In some embodiments, the transaction recording module verifies the NDC of the kit being added by analyzing the image of the sensitive medication pack. In some embodiments, a unique ID encoded in a tag and available on the sensitive medication pack is recorded. The transaction recording module is used to record all changes made to kits and provides details of all contents for a pharmacist to review and approve the kit prior to providing the kit to a clinical user for administration to a patient. In some embodiments, the transaction recording module records transaction as related to direct retrieval of sensitive medication packs from a storage.

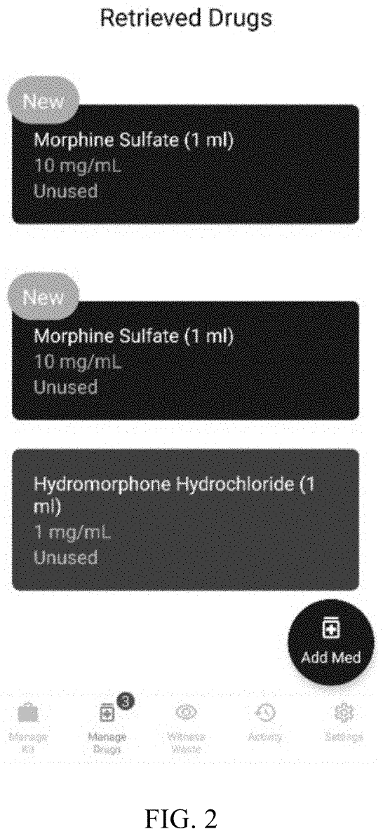

In some embodiments, the transaction recording module records the retrieval of sensitive medication packs from a certain drug container such as a medical kit or from an ADC and displays this to the user through the transaction recording GUI. FIG. 2 illustrates an embodiment of the transaction recording GUI related to drugs retrieved from a kit as part of a mobile application for a smartphone.

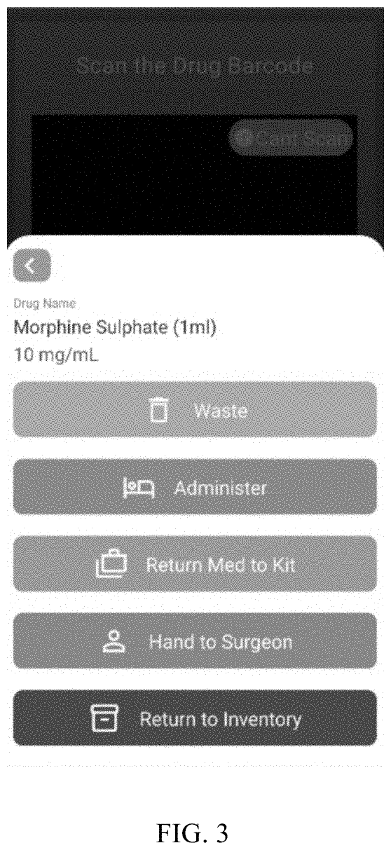

In some embodiments, the transaction recording module records administration, waste, return to the medical kit, transfer to a second person (e.g., a surgeon) or return to inventory of a specific drug and displays this to the user through the transaction recording GUI. FIG. 3 illustrates the exemplary embodiments of the transaction recording GUI related to administration, waste, return to the medical kit, transfer to a second person (e.g., a surgeon) or return to inventory of a specific drug (e.g., morphine sulphate) as part of a mobile application for a smartphone.

Request for Witnessing an Event

The transaction recording module has the capability to record a transaction involving more than one user. An example application involves the witness process for wasting a controlled substance. In this process, a first user requires a witness to waste (discard) a controlled substance. The transaction recording module captures data for the waste action, including the identifiers for the drug being wasted (name, NDC, volume, mass, concentration), and the time and date and location where waste is happening. To record a waste action involving a witness, one or more transaction recording modules and one or more image and video processing modules are used. Here, we refer to the user intending to waste their drug as the wasting user and the person serving as the witness as the witness user.

In some embodiments, the wasting user and the witness user use their respective transaction recording modules and image and video processing modules. In some embodiments, the transaction recording module and the image and video processing module runs on one smartphone and the user intending to waste their drug and the witness use their respective smartphone.

First, a witness waste request code is generated by the wasting user's transaction recording module. This witness waste code is transmitted to the witness user's transaction recording module. In some embodiments, a remote server or local Bluetooth communication or near field communication may be used for such transmission. In some embodiments, the witness waste request code is encoded in a barcode displayed on the graphical user interface of the wasting user's transaction recording module and scanned by the image and video processing module of the witness user.

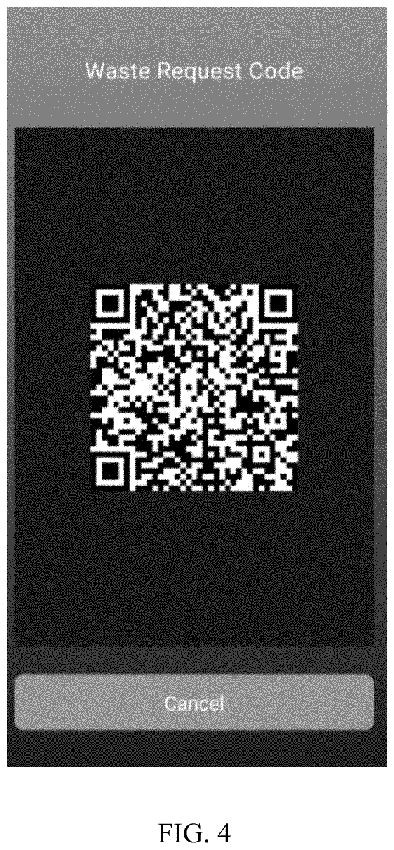

In some embodiments, a witness waste request barcode is generated by the transaction recording module of the wasting user to embed the information related to the waste action. Examples of a witness waste barcode is a QR code or datamatrix or any other 2D or linear barcode. FIG. 4 illustrates an example witness waste request barcode in the form of a “waste request” QR code which is displayed on a transaction recording module implemented on as a mobile application on a smartphone.

Once the witness waste request code is received by the witness user transaction recording module and the witness user approves the waste, a witness waste confirmation code is generated by the witness user's transaction recording module. This witness waste confirmation code is transmitted to the witness user's transaction recording module. In some embodiments, a remote server or local Bluetooth communication or near field communication may be used for such transmission. In some embodiments, the witness waste confirmation code is encoded in a barcode displayed on the graphical user interface of the witness user's transaction recording module and scanned by the image and video processing module of the waster user. In some embodiments, only one image and video processing module exists which is used to scan the witness waste request code and witness waste confirmation code.

Each user of the transaction recording module (including the witness user and the wasting user) may authenticate their identity using a biometric-based authentication method or a username and password. When a user attempts to login, the database module compares the username and password (or biometric-based information) against that user's authentication credentials and if they match, the database module generates an access token using the SH256 algorithm SH512, or another appropriate hash algorithm. This token is used to identify and authorize any user application programming interface (API) requests to the database module.

An example access token is a JSON web token. After the witness user enters their final decision (e.g., to accept/reject a waste request) using the transaction recording module, the transaction recording module of the witness user transmits the access token and the final decision to the database module. The wasting user enters their access token and waste action details such as identifiers for the drug being wasted (such as name, NDC, volume, mass, concentration), and the time and date and location where waste is happening to the database module.

The database module decodes the access token for the witness user and the wasting user using their respective private key stored on the database module. If this decoding process is successful, then the database module records this waste event and all related data such as the user's identities, identifiers for the drug being wasted (name, NDC, volume, mass, concentration), and the time and date and location where waste is happening, in a database. If a token is tampered with in any way or expired, the decoding fails.

This process can address issues with lack of internet connectivity. The database module may receive the required information from one user and not the other. In such scenario, a waste request process remains pending when one user is successfully authorized and but the other user is still awaiting an internet connection. Once the required information from both users have been received by the database module, the recording process for the waste is completed.

In some embodiments, the system for recording waste by a witness does not include a computation engine module. In this embodiment, the system comprises one or more transaction recording modules, a database module, one or more image and video processing modules. In some embodiments, a computation engine module is part of the system, wherein the computation engine module is responsible for reviewing witness transaction data and identify potential fraudulent activities.

Database Module

The database module communicates with one or more transaction recording modules and one or more image and video processing modules and the computation engine module. In some embodiments, the database module stores at least some transaction information in an untamperable read-only non-relational database. In some embodiments, transactions are stored in a blockchain journal that may be audited. The Amazon QLDB (Quantum ledger database) is an example of such a database.

The database module stores information in a series of database tables comprising tables related to users, activity, medical kits, drugs, drug information, inventory, inventory locations, and waste requests among others. In an exemplary embodiment, an operation may involve adding a drug to a medical kit. This involves retrieving relevant information about the drug based on the NDC from a secondary database (e.g., the drug information database maintained by the FDA) and recording this retrieved information in the drug information table. Then a record is written to the drug table that contains the quantity of the drug being added, its NDC number, and the unique id of the kit it is being added to. Actions undertaken by a user are always recorded with a timestamp in the activity table. Examples include retrieving a kit, removing a drug, administering a drug, wasting a drug, witnessing a waste, auditing inventory, etc.

In some embodiments, a read-only ledger database such as QLDB exists as a source of truth that other services can copy from or check against. Data is always ingested into the read-only database first and then copied to a secondary replicated database afterwards. The application contains error handling logic to catch any transaction failures in the secondary database and resolves them against the source of truth database. A replicated database such as Amazon DynamoDB is utilized because complex queries against the main datastore (Amazon QLDB) may be too slow for ideal real-time use or are unsupported altogether. As an example, the “Outer Join” statement and “Limit” SQL statements are not supported by QLDB and these limitations are addressed by a replicated database.

The database module is responsible for authenticating users. This is done through its user verification and access token generation subcomponent. To verify the identity of the user, the following data is captured by the transaction recording module and transmitted to the database module: i) a biometric verification process such as fingerprint scanning or facial feature scanning which is built-in to the device or as a separate device; and/or ii) a username and password combination which can be verified by the user verification and access token generation subcomponent with a previously recorded username and password for that user recorded the database.

All users using their respective transaction recording module are required to be authenticated. Upon authentication, the database module generates a unique access token for each user. In some embodiments, a user name and password combination is used to authenticate a user. In some embodiments, biometric-based authentication methods such as finger print or facial feature scans are used to authenticate users.

In some embodiments, the database module is responsible for keeping track of the inventory related to sensitive medication. This includes computing the current quantity of various types of sensitive medication in different locations of a healthcare facility based on recorded transactions related to adding or removal of sensitive medication packs. In some embodiments, the database module can generate a report on current inventory to assist with a count of sensitive medication packs in the inventory.

Image and Video Processing Module

The disclosed invention also includes an image and video capture module. In some embodiments, the image and video capture module is a smartphone camera and its associated application, a camera mounted on the wall, a wearable camera such as an augmented reality headset or augmented reality glasses, or a camera worn on the body. In some embodiments, administration of a sensitive medication to the patient can be documented by scanning the identification tag of the sensitive medication packs.

The image and video processing module comprises one or more subcomponents as follows: 1) an image and video recording subcomponent which is responsible for capturing and recording the image and/or video; and 2) an image and video analysis subcomponent to analyze the contents of the image and extract alphanumeric characters and other meaningful information from printed text on labels on sensitive medication packs such as drug name, NDC, expiration date, lot number, serial number, and detect whether the sensitive medication pack is actually used after the user confirms its use. Verification of whether the drug was used is important for cases where theft may be an issue, such as diversion of controlled substances. The image and video analysis module can use computer vision techniques such as optical character recognition (OCR) or other computer vision methods to detect alphanumeric characters and automatically extract information from printed labels and hard copy material. Examples include reading NDC, expiration date, lot number, serial number, and quantity, among others. In some embodiments, information from a hardcopy document such as an invoice is used to extract information regarding batch, box, or shipment of sensitive medication packs that is then input into the system.

In some embodiments, the image and video analysis uses standard techniques for reading or scanning linear and 2D barcodes including datamatrix, databar, and other standard barcodes to extract information encoded in such barcodes. In some embodiments, information extracted from sensitive medication packs are communicated with the transaction recording module directly. In some embodiments, the information extracted from the image and/or video is communicated with the database module directly. In some embodiments, the information extracted from the image and/or video is communicated with the database module and transaction recording module. In some embodiments, the captured video and/or image is communicated with the database module to record such video and/or image for future review.

In some embodiments, a picture of the sensitive medication pack, after use, can be recorded by the image and video processing module after administration or after wasting (discarding) the sensitive medication. In some embodiments, an image of a removed pill from a blister pack may be recorded as evidence of administration of the sensitive medication to a patient. In some embodiments, the image and video capture module is used to record the process of administration of the sensitive medication or the process of wasting (or discarding) the sensitive medication. In such embodiments, the video shows the process of administration of the sensitive medication to the patient or shows the process of wasting or discarding the sensitive medication.

In some embodiments, all images and videos are assigned a unique identification number. The identification number and the transaction details including the user, current location, current time and date, and identification tag associated with the sensitive medication pack, are recorded in the database to generate an audit trail. The recorded image and/or video is also recorded on the same database or, optionally, a second database. In the case where the image and/or video is recorded on a second database, the unique identification number of the image and/or video is recorded in the second database and the unique identification number is also recorded in the first database as part of the transaction data such that the correspondence between the transaction and the image and/or video can be established across databases.

In some embodiments, the sensitive medication is scanned and/or photographed and/or a video is recorded as evidence that the sensitive medication was used, administered, or wasted. In some embodiments, an automated image or video processing framework may be used to verify that the sensitive medication pack was actually used. In some embodiments, an image classification algorithm such as a neural network is used to classify sensitive medication packs into two classes: “used” and “not used.”

In some embodiments, features from the image such as color or shape (missing liquid inside the vial or ampule, deformation in shape such as holes, breaks, or punctures in packaging) of the sensitive medication pack is used to classify packs. In some embodiments, a missing component of the sensitive medication pack is used as a feature for classification. Example features are, a missing cap or tip of an ampule or vial. In order to perform automated classification of sensitive medication packs, a machine learning classifier is used as the image classification algorithm.

Machine learning classifiers used for classification include one or more of the following method or a combination thereof: classification and regression trees (CART), random forests, bagging classifier, boosting algorithms such as AdaBoost, RealAdaBoost, among others, support vector machine (SVM), naive Bayes classifier, k-nearest neighbor, Bayesian networks, neural networks including deep neural networks, convolutional neural networks, recurrent neural networks or the like as are known and accepted in the art.

Computation Engine Module

The disclosed invention can be used to detect potentially fraudulent activities or policy violations for handling sensitive medication. An exemplary fraudulent activity is drug diversion involving a controlled substance. In this disclosure, fraudulent activity, potentially fraudulent activity, policy violation, red flag, and outlier are used interchangeably and refer to events and or transactions that need to be further investigated as they may violate a policy or law related to handling, use, transportation, disposal, and/or administration of sensitive medication. An exemplary embodiment of policy or legal violations is drug diversion related to controlled substances.

Throughout this disclosure, potentially fraudulent activity is a general term encompassing potential fraudulent activity, policy or legal violations, or a transaction that is considered an outlier and needs to be further investigated.

A computation engine is responsible for analysis of transactions recorded in the database module and identifying potentially fraudulent activities and policy or legal violations. In some embodiments, the computation engine also uses information received from other sources such as the electronic medical record (EMR) and the automated dispensing cabinets (in addition to data received from the database) to detect potentially fraudulent activity (e.g., drug diversion). Information received from external sources include patient name and/or identification number (medical record number, or other identifier), drug name, concentration, administration amount mass and/or volume, time and date of event, location of event, the person and/or persons involved in the event, any manual overrides, and other information routinely recorded in the EMR or the automated dispensing cabinet. In some embodiments, potentially fraudulent activity is detected and recorded in the database module. In some embodiments, notification of a detected potentially fraudulent activity is sent using a text message, an automated call, an email, a pop-up window in a software application, or a smartphone push notification message or other types of standard notifications used in mobile or web applications to the appropriate users with the appropriate roles and data access within the system.

In some embodiments, the computation engine creates an audit trail for reviewing the chain of custody of a one or more sensitive medication packs. An audit trail involves transaction data associated with sensitive medication packs comprising user identity, time and date of scanned tags, recorded locations at different time points, the kits (if any) or storage location (if any) where the medication was retrieved, all persons who handled or were involved in one of the transactions, time, date, and location of administration to the patient and patient's identification tag (if any) or, if discarded, the user identity, time, date and location of the discarding process (i.e., referred to as waste).

The computation engine can assist in reviewing and auditing purposes as well. The computation engine can retrieve transaction data, and/or data received from the image and video processing module. In some embodiments, an auditor may request to review evidence of administration of one or more sensitive medication packs or evidence that one or more sensitive medication packs have been used or wasted. The computation engine can retrieve image and/or video of a used or empty sensitive medication pack or a video of the administration process of a sensitive medication pack from the database. Such retrieved image and/or data can be used for audit purposes.

The computation engine can generate an audit trail associated with the scanned tag. The audit trail includes all historical transactions associated with one or more sensitive medication packs including the various locations the sensitive medication has been stored, the identity of person or persons moving the sensitive medication pack from one location to another or from a first person to a second person, and the timing of the events.

The computation engine module is composed of three subcomponents: 1) a transaction analysis subcomponent; 2) a computation engine graphical user interface; and 3) a notification subcomponent.

The transaction analysis subcomponent is responsible for reviewing recorded transactions to detect the following: a) potential fraud activity or policy violations; and b) any activity that requires the attention of a user such as approving a kit or serving as a witness.

The computation engine GUI is used to show retrieved data from the database, allow the user to generate queries from the database, and communicate information generated or identified by the computation engine with the user. In some embodiments, the computation engine GUI is implemented as a web application and the user can access database information and create various queries involving transactions such as transactions based on kit, based on a specific user, based on a specific drug among others. In some embodiments, the computation engine GUI is used to generate a report for a specific facility such as a report for all drug wastes, a report for expired medications, a report for a user's activity over a period of time, among others.

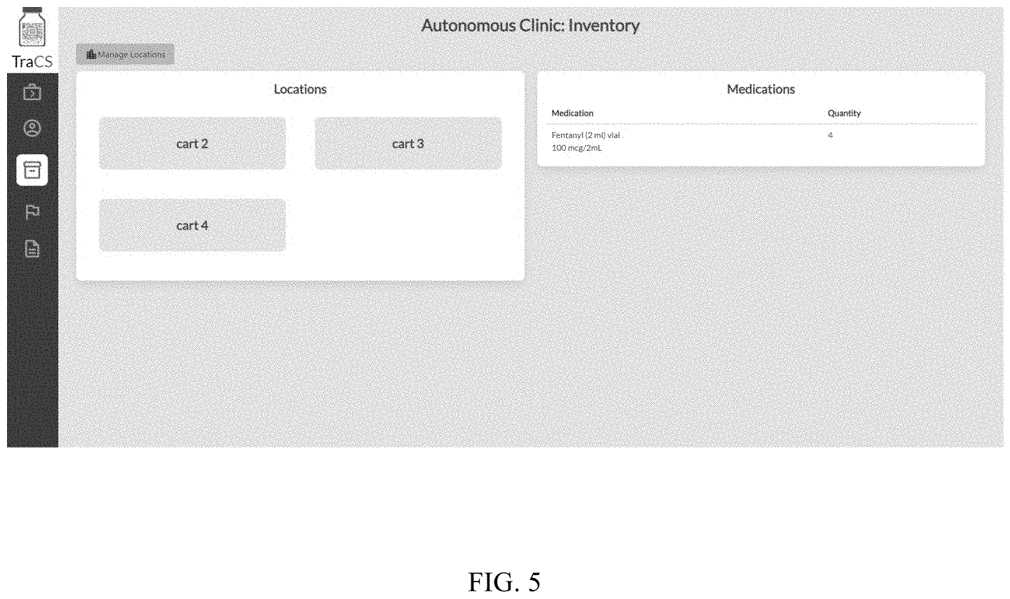

In some embodiments, the computation engine can retrieve current inventory at different locations from the database module and display to the user through the computation engine GUI. FIG. 5 illustrates embodiments of the computation engine GUI related to current inventory at different locations.

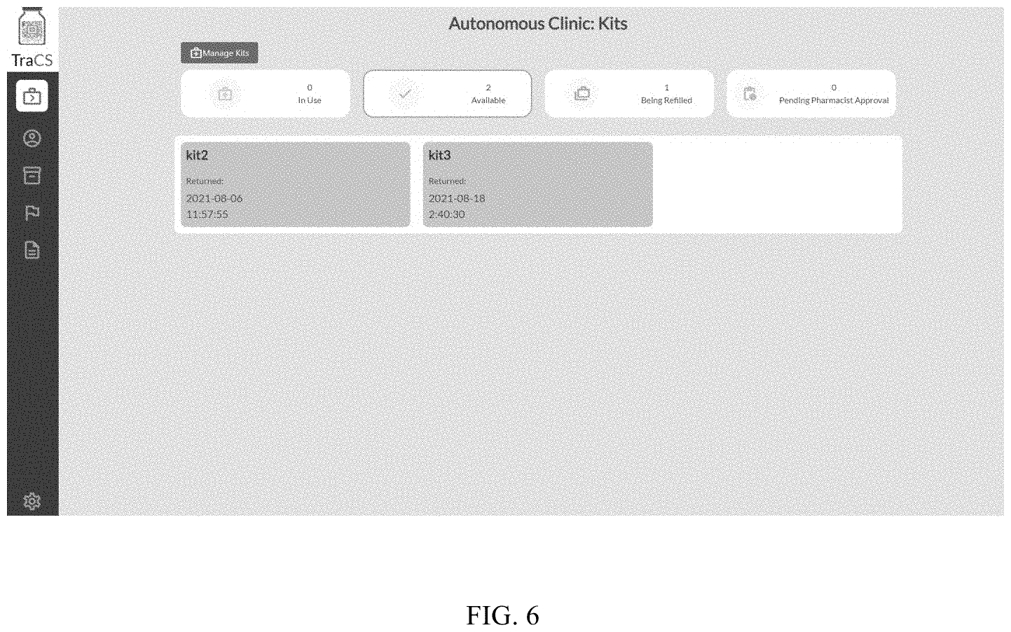

In some embodiments, the computation engine can retrieve current status of medical kits from the database module and display to the user through the computation engine graphical user interface. FIG. 6 illustrates embodiments of the computation engine GUI related to displaying the current status of medical kits.

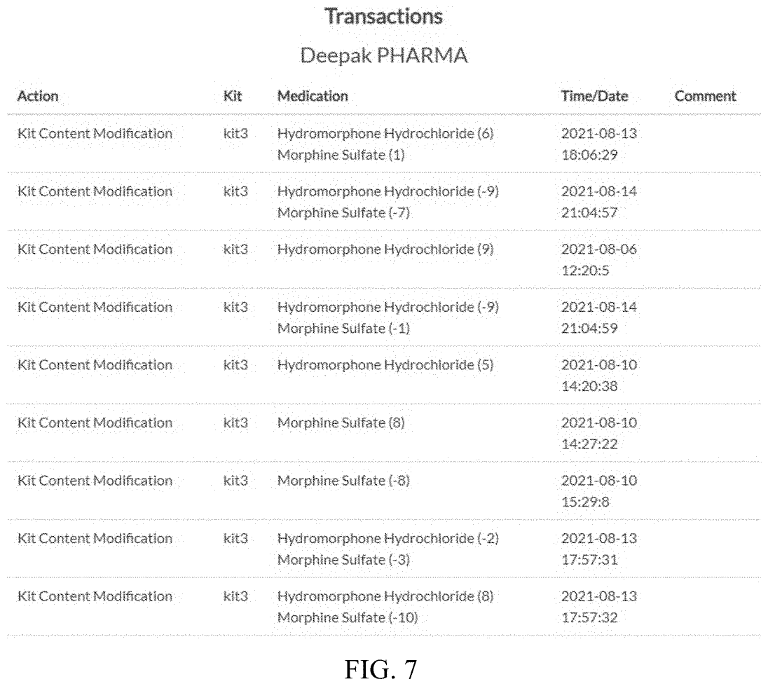

In some embodiments, the computation engine can retrieve transactions associated with a specific user from the database module and display to the user through the computation engine graphical user interface. FIG. 7 illustrates the exemplary embodiments of the computation engine graphical user interface related to displaying transactions associated with a user “Deepak PHARMA”.

The notification subcomponent is responsible to communicate information that is time sensitive or requires the attention of the user. This notification is sent using a text message, an automated call, an email, a pop-up window in a software application, or a smartphone push notification message or other types of standard notifications used in mobile or web applications.

Transaction Analysis Subcomponent

The transaction analysis subcomponent has a fraud detection algorithm (classifier) responsible for finding potentially fraudulent activities. In some embodiments, a machine learning classifier is used. In some embodiments, a rule-based classifier is used. In some embodiments, a combination of a machine learning classifier and a rule-based classifier is used. In some embodiments, the machine learning classifier is trained on real transaction data obtained from a facility. In some embodiments, a simulation is used to generate synthetic data to train the machine learning classifier. The synthetic data is used to artificially introduce fraudulent activity and policy or legal violations in order to train a machine learning classifier.

Synthetic Data Generation

In some embodiments a discrete-event simulation, based on a discrete-time Markov chain, is used in order to generate synthetic datasets, closely resembling real-world data. The simulation includes multiple agents of different roles (such as pharmacists and anesthetists), each associated with a unique Markov chain, allowing transitions between different states, with each state denoting an action allowed for that specific role, and each transition signaling switching between one action to another (or repeating the same action; i.e., self-transition). Thus, each transition is considered the beginning of a new action (e.g., refilling a drug kit by a pharmacist, or administering a drug to a patient by an anesthetist), and is registered in a log similarly to the way such actions are registered when using the application. The simulated actions of various agents using this framework generates synthetic transactions, which can be recorded in a database or log file and used for training machine learning classifiers.

The simulation also incorporates multiple objects, including drugs, drug kits and automated dispensing cabinets (ADCs), having unique properties and states, determined by actions performed by agents on those objects (e.g., a drug vial is moved from inventory to a kit which is being refilled by a pharmacy technician; then, the kit is approved by a pharmacist, and is next picked up by an anesthetist, which loads it into a syringe, and finally administers it to a patient or wastes the drug).

The simulation outputs a log file, containing all actions performed by the different agents and corresponding information, such as time action started and performing agent identity, as well as action-specific information where relevant, such as the type and amount of drug administered by an anesthetist to a patient.

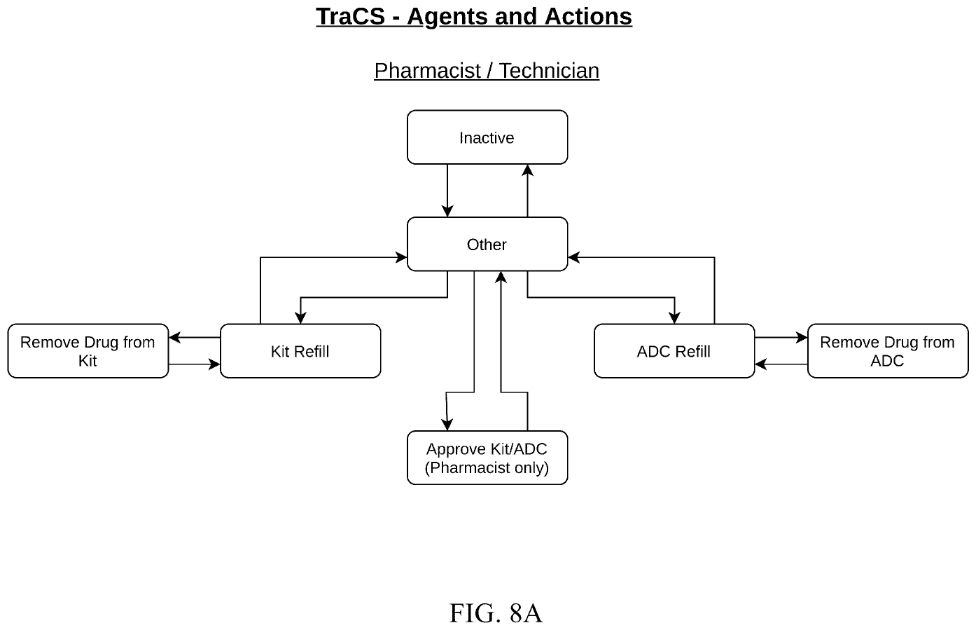

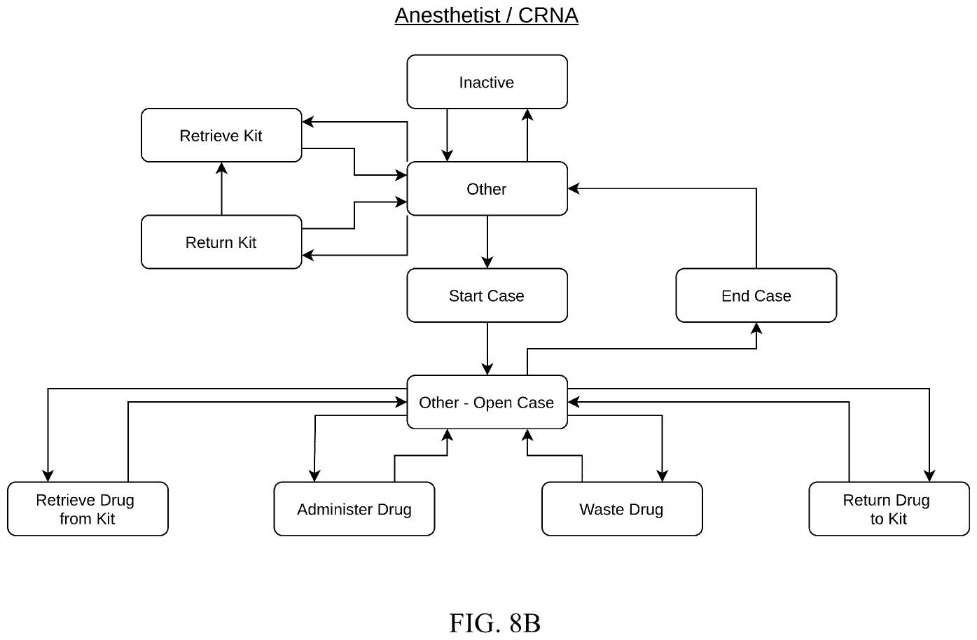

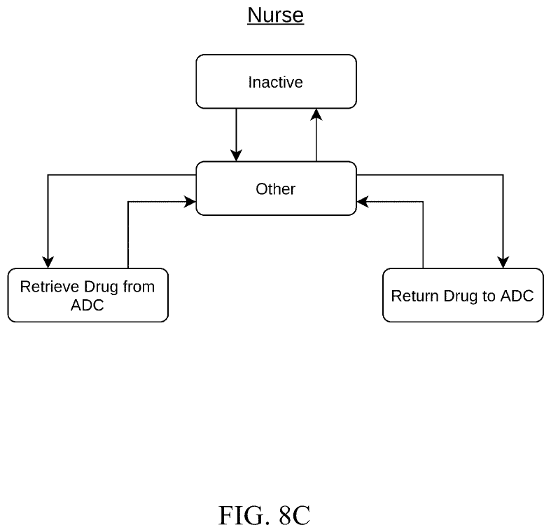

FIGS. 8A-C illustrate an example of state diagrams of Markov chains corresponding to different roles used within the simulation. In the figures, each state (rectangle) represents an action the simulation agent is engaged in at a given time, while transitions (arrows) represent switching between one action to another. Actions can be repeated multiple times (i.e., self-transitions are allowed). When not engaged in an active shift, agents are kept in the “Inactive” state.

Synthetic Data Generation through Simulation

Below is an example synthetic data generation activity. The simulation is designed to closely imitate real-world behavior of human users of the application. Synthetic datasets generated via the simulation can be used to develop and test a fraud detection system as part of the transaction analysis subcomponent of the computation engine. Importantly, such datasets can include known occurrences of different fraudulent actions (such that each action can have an associated “fraud” and “not fraud” label), which can be very challenging to gather from real-world data. Furthermore, if a machine learning classifier is used for the fraud detection system, the classifier is trained on synthetic data and tested on real-world data collected from users of the technology.

Models trained based on synthetic data can optionally serve as a “warm-start”, while further tuning (or training or re-training) can be done on additionally gathered data, from real-world use of the technology. For example, a machine learning classifier is trained on synthetic data can be further retrained by combining synthetic data with real-world data collected from the use of the technology (and recording relevant transactions) for a specific facility. Such combination of synthetic and real-world data allows for creating “customized” classifiers for a specific facility.

The simulation program includes multiple parameters, allowing it to represent different hospitals, or any other facility. These include, but not limited to: shift schedule; roles of application users (e.g., pharmacist, pharmacy technician and anesthetist) and their numbers; types of objects (e.g., drug containers or medical kits or ADC); kit quantity, and distribution between users (e.g., two kits are associated with each anesthetist; facility has one ADC); drug container refill schedules (e.g., drug kits are returned to be refilled when having less than a random percentage in the range of 35-75% of the capacity of one or more drugs; ADCs are filled twice a day at specific hours); types of drugs used, their available quantities and/or number of drug containers (e.g., vials) available in each type of drug storage (e.g., kit and/or ADC); drug storage capacity (e.g., number of vials, amps, syringes and/or carpujects of each drug type in drug kits and in ADCs); probabilities and/or minimum and maximum amounts of drug included in a single administration for each drug type; probabilities of each drug being chosen for administration; types of possible fraudulent actions.

At the beginning of each week, a shift schedule is automatically determined for each agent according to predefined shift hours, with the number of agents of each role in each shift balanced across shifts. Agents begin in an “inactive” state, and transition into the “other” state when a shift begins, and when it ends. The simulation time moves forward between a predefined start and end times, in a fixed and predefined increment (i.e., “time step”). At each time step, every available agent (who is not currently engaged in an action) “choses” to perform a new action randomly from all possible actions for his/her current state, according to a preset probability of each of these actions.

Actions related to drug transportation, administration, and waste are logged in an array (also referred to as a data frame), together with its timing and other information comprising the specific details of the action (e.g., amount of drug administered to a patient), the agent or agents involved in performing the action (e.g., an anesthetist wasting a syringe partially filled with a drug and a witness to this action), and the objects (e.g., drug vial, kit or syringe) involved in the action. Other non-relevant actions are labeled as “other” actions and are not considered.

Every time an action is initiated, the agent transitions between the states corresponding to the last and new actions, respectively, and enters a queue for a given number of time steps, until the action is “completed”, and then he/she “chooses” a new action, or ends the current shift and enters an “inactive” state. While each action is associated with a specific duration (i.e., number of time steps) and probability to be engaged with, some variations are introduced (to create a more realistic dataset). For example, variations are introduced by multiplying the values by weights randomly sampled for each agent in some predefined range (e.g., from a normal distribution, in the range of 0.5-1.5).

The simulation program also includes fraudulent actions. A predefined fraction of agents of each role has a chance to engage in fraud, each randomly assigned with a specific probability to engage in fraudulent activity, which, for example, is randomly assigned from some predefined distribution in a predefined range (e.g., a normal distribution in the range of 0.01-0.05). Every time one of these agents engages in an action which has an alternative fraudulent action, he/she performs instead a fraudulent action—either intentionally or by mistake. Moreover, different fraudulent actions can potentially have different probabilities to be committed intentionally or by an operational error; these can also differ from one agent to another. To create these variations in probability, each agent is randomly assigned a probability at random, according to some distribution and range.

Probabilities mentioned above, relating to actions choice, duration, fraud, error, or any other element of the simulation, can be fixed or dynamically changed by certain factors, including, but not limited to, time of day, agent individual characteristics (e.g., age and/or gender), among others.

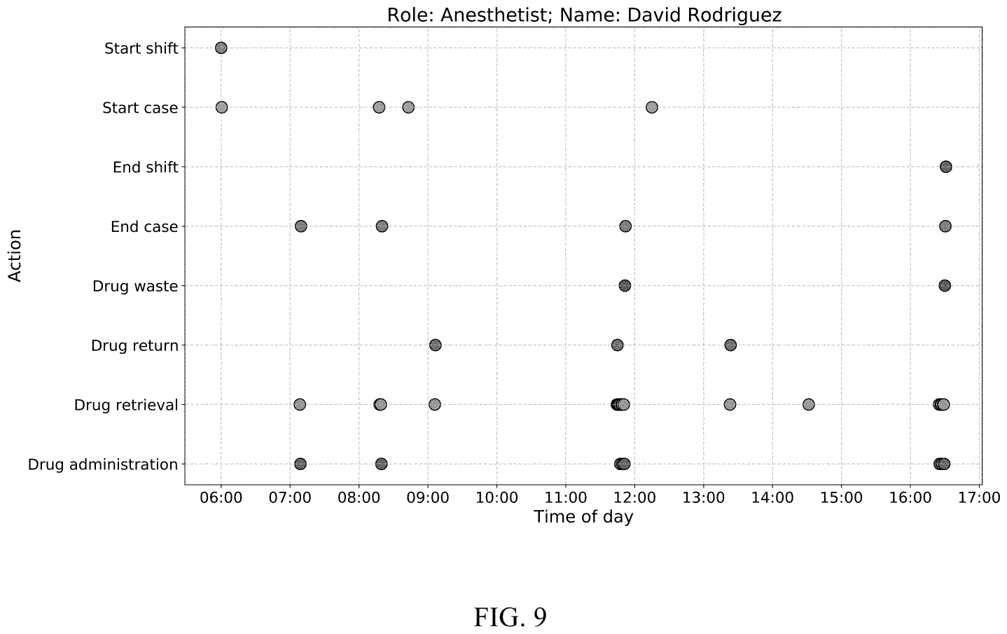

Data was generated using a simulation of an ambulatory surgical center. A full year of data was synthetically generated in the time period between August 2021 and August 2022. Time step size was set to 30 seconds (1,051,200 time steps overall). 4 pharmacists, 3 pharmacy technicians, 15 anesthetists and 45 nurses were included in the simulation. 30 drug kits were distributed among the anesthetists (a maximum of 2 kits for each at each given time; see Table 1), and one ADC was used by nurses to retrieve drugs from. A total of 169,369 actions were recorded in the simulation. FIG. 9 illustrates an example single-shift timeline, indicating each action logged for a user with an anesthetist role.

TABLE 1
Example of the capacity of a single drug kit, as used in the
example simulation.

| | Amount in a single container | |
|---|---|---|
| Drug type | (ml; undiluted) | Container quantity |
| Fentanyl | 5 | 5 |
| Fentanyl | 2 | 20 |
| Morphine | 1 | 5 |
| Versed | 2 | 10 |
| Ketamine | 10 | 5 |
| Dilaudid | 1 | 5 |
| Demerol | 1 | 5 |
| Alfenta | 2 | 5 |

Detection of Potentially Fraudulent Activities

The synthetic dataset generated by the simulation can be used to develop a fraud detection algorithm to identify potentially fraudulent activities and policy or legal violations. The problem such an algorithm is solving is a classification problem: predicting whether a transaction is potentially fraudulent or not, and/or providing an estimation of the probability of an action being fraudulent. To achieve this, the algorithm computes a decision score and relies on one or more pre-determined decision thresholds, such that the prediction it makes depends on whether or not a single, or multiple values of the decision score exceed one or more pre-determined decision thresholds.

For example, one or more decision scores can be calculated for a given transaction by a single or multiple classifiers, and the action would be flagged as potentially fraudulent if its associated decision score exceeds a pre-determined decision threshold, and/or if some or most of the multiple decision scores exceed their corresponding pre-determined decision thresholds. Thus, the fraud detection algorithm may involve, but not be limited to, one or more static rule-based algorithms, and/or machine-learning algorithms, and/or data mining models, and/or any other types of mathematical models, which involves computing a decision score associated with a transaction and comparing the decision score with a pre-determined decision threshold to determine if the transaction needs to be classified as a potential fraudulent activity.

Static rule-based algorithms involve a predefined set of if-then rules—created based on domain knowledge—against which every new transaction registered in the application is examined to estimate the probability of it being fraudulent. For example, one or more decision tree algorithms can be used, which involve a decision rule at each node, splitting between two or more branches based on the features, and repeating this process until a final leaf node is reached, signaling a decision (e.g., “normal” or “fraudulent” transaction). The decision rules can be formalized based on expert knowledge, and/or learned from the data.

A machine-learning classifier (or classification algorithm) involves a single model, or an ensemble of models, which learn or estimate their parameters from the data during a training period, with the goal of capturing the mapping between input and output (e.g., between features related to a single transaction and a legitimate/fraudulent label, or a probability of fraudulent activity). Machine learning models typically require a large dataset for proper training, which can be generated by the simulation, and later fine-tuned for specific locations in production.

A machine learning algorithm has to first be trained. Once the model is trained, the trained model can be used within the computation engine to analyze transactions. A machine learning algorithm is first initialized with a specific set of preset hyper-parameters, which control the learning process (e.g., learning rate, choice of optimization algorithm, and loss function); the learning process itself involves tuning the model parameters to optimize its performance for the training set. Next, multiple trained algorithms, involving different sets of hyperparameters and/or algorithm types, are typically evaluated on a separate validation set, containing samples not included in the training set, and then their performances are compared in order to obtain the optimal model.

While a single, independent validation set can be used, a common approach is to use cross-validation, in which the training data is divided into n subsets (for example 2, 3, 4 5, 10, 20), and for each subset, the model is trained on the n−1 remaining subsets, and then validated on the remaining subset; finally, an overall validation score is calculated for a given model based on the results obtained for all the subsets. A common approach to model optimization involves training and validating multiple models having different sets of hyperparameters, then choosing one or more models having the highest validation scores. Finally, models are typically examined again on a separate test set. Models can be scored using different evaluation methods, including and not limited to, precision and recall, sensitivity and specificity, accuracy, and/or Cohen's Kappa.

The goal of the fraud detection algorithm is to monitor transactions recorded in the database by analyzing individual transactions, or batches of transactions, either online and/or offline, and alert the administrator or other user with an appropriate role for receipt of such possible fraud detection alerts if one or more actions are found to be a potentially fraudulent activity. Specifically, different types of transactions have various properties which can be used as a group of features for the machine learning model input with the goal of identifying and reporting any abnormalities based on these features (see Table 2). The fraud detection algorithm may include one, or any combination of the above-mentioned model types, including mathematical models of similar nature and not detailed here. Suspicious transactions, that is, transactions classified as potentially fraudulent activity are flagged as possibly fraudulent, and the final decision of whether or not to consider an action as fraudulent is up to the system administrator once an investigation and/or review is performed.

In some embodiments, the administrator can have control over which actions are considered potentially fraudulent for their specific facility by creating custom if-then rules or designating certain transactions as fraudulent for further training the fraud-detection model. In some embodiments, any post-editing of reported transactions in the application by the user after the information is sent may be flagged as potentially fraudulent.

Features which can be extracted from transactions are shown in Table 2. Drug storage refers to a kit, an ADC, or any other tool or location used for storing medication packs (including, but not limited to vials, ampules, and carpujects).

TABLE 2
Features which can be extracted from transactions related to sensitive
medications

| Action | Example scenario | Features |
|---|---|---|
| Drug retrieval | An anesthetist retrieves a | Drug type; drug amount; |
| | drug vial from his/her kit | number of previous |
| | | administrations of same drug |
| | | in current case; number of |
| | | total drug administrations in |
| | | current case; time until drug |
| | | expiration date; time since |
| | | drug was added to storage; |
| | | time since drug was added to |
| | | inventory; number of drugs |
| | | currently in storage; time |
| | | since storage last refill; time |
| | | of day; action duration; time |
| | | since shift start |
| Drug administration | An anesthetist administers a | Drug type; amount of drug |
| | drug he/she retrieved | administered; amount of drug |
| | | remaining in syringe; number |
| | | of previous administrations of |
| | | same drug in current case; |
| | | number of total drug |
| | | administrations in current |
| | | case; time of day; action |
| | | duration; time since shift start |
| Drug return | An anesthetist returns a | Drug type; amount of drug; |
| | previously retrieved drug vial | storage type (e.g., kit/ADC); |
| | back to his/her kit; a nurse | person serving as witness |
| | returns a drug vial to an ADC | (i.e., witness id); witness role; |
| | | ratio of times same person |
| | | served as a witness; ratio of |
| | | time user served as a witness |
| | | for the current witness; time |
| | | until drug expiration date; |
| | | time since drug was added to |
| | | storage; time since drug was |
| | | added to inventory; number |
| | | of drugs currently in storage; |
| | | time since storage last refill; |
| | | number of previous |
| | | administrations of same drug |
| | | in current case; number of |
| | | total drug administrations in |
| | | current case; time of day; |
| | | action duration; time since |
| | | shift start |
| Drug waste | An anesthetist sends a | Drug type; amount of drug |
| | partially-full syringe to be | wasted; Witness id; witness |
| | wasted after he/she used it to | role; ratio of times same |
| | administer a drug to a patient | person served as a witness; |
| | once or multiple times | ratio of time user served as a |
| | | witness for the current |
| | | witness; number of previous |
| | | administrations of same drug |
| | | in current case; number of |
| | | total drug administrations in |
| | | current case; time of day; |
| | | action duration; time since |
| | | shift start |
| Drug storage refill/ drug | A pharmacist, or pharmacy | container capacity before |
| removal from storage | technician, refills a kit, ADC, | refill; container capacity after |
| | or any other type of drug | refill; number of missing |
| | container, while also | units per type before refill; |
| | checking its content and | number of missing units per |
| | removing drug containers | type after refill; |
| | due to reasons such as being | time since storage last refill; |
| | close to or surpassing | time of day; action duration; |
| | expiration date, a recall | time since shift start |
| | initiated by the manufacturer | |
| Kit retrieval | An anesthetist retrieves a | Kit last refill time and date; |
| | new drug kit from pharmacy | current number of drug |
| | | containers in kit |

In some embodiments, each transaction is first be examined using a static rule-based model, such as a decision tree to detect a potential fraudulent activity; and then, the same transaction is analyzed by one or more machine learning classifiers, and/or other types of models, and the transaction will be considered a potential fraudulent activity if found to be potentially fraudulent by one machine learning classifier (also referred to as a machine learning model), a number of machine models, and/or according to an overall decision score calculated from multiple models' outputs.

In an alternate embodiment, each transaction is analyzed, in sequence or in parallel, using two or more machine learning models, while the final decision whether the action should be classified as a potentially fraudulent activity would be determined by a combination of each model's decision; for example, for a multi-model classifier, an overall decision score may be computed through a weighted sum. For example, for a 3-model classifier the classification is performed by calculating w1*d1+w2*d2+w3*d3, such that if the weighted sum w1*d1+w2*d2+w3*d3>1 then the output class is 1, otherwise 0, where each out of three models is represented by a different number (1,2,3), d denotes a model's decision score, and w represents a weight associated with a specific model's decision, and Σᵢ₌₁³wᵢ=1.

Static Rule-Based Models

A static rule-based model, such as a decision tree, can be designed based on expert knowledge and used to determine whether a given action is potentially fraudulent based on one or more features relevant to that action. An exemplary embodiment of a decision tree involves evaluating whether nw=nr after an anesthetist user has reported ending a case, where nw indicates the number of wasted drug syringes, and nr corresponds to the number of types of drugs retrieved; if nw=nr is not satisfied (i.e., nw≠nr), then related transactions are labeled (classified) as potential fraudulent activity, and the administrator or other relevant user role is notified through the notification subcomponent.

In some embodiments, if an anesthetist has drawn vials of three different types in a single case, but only reported the waste of syringes holding two types of drugs (i.e., nw<nr), then these waste actions can be flagged as potentially fraudulent, since the user could potentially divert the unwasted drug (a single syringe cannot hold more than one type of drug).

Machine Learning Classifiers

Another approach to detecting potentially fraudulent activities is the use of one or more machine learning algorithms, able to learn from the data by adjusting their parameters to optimize model performance. In some embodiments, one or more of machine learning algorithms, having varying sets of hyperparameters, will be trained and evaluated on synthetic data generated by the simulation, and further tuned as required to optimize performance. In some embodiments, one or more machine learning algorithms are trained on real data obtained through externally-sourced data collection or through the regular use of the system. Next, optimal models will be tested on a test set composed of real-world data, such as data collected from use of the overall system and/or from an external source, including, but not limited to, an electronic medical record (EMR).

One or a combination of two or more of the following approaches is used as machine learning algorithms to detect potentially fraudulent activities. In some embodiments, labeled data exists for training machine learning classifiers. If labeled data exist (i.e., through a training set, we have access to transactions that are labeled as “fraud” and those labeled as “not fraud”), then machine learning algorithms such as, but not limited to, logistic regression, random forest, support vector machines (SVMs), boosting algorithms such as gradient boosting and AdaBoost, and neural networks are used.

In some embodiments, labeled data does not exist (i.e., unlabeled dataset) and algorithms such as, but not limited to, isolation forest, and/or neural autoencoder, are used for outlier and anomaly detection (e.g., for flagging outlier cases, or anomalies, as potentially fraudulent activities).

In some embodiments, the risk of missing a fraudulent action (i.e., false negative, or type II error) is considerably higher than the risk of producing an erroneous alert for fraud (i.e., false positive, or type I error). In this case, a conservative approach would be to minimize the probability of false negatives at the expense of increasing the probability of false positives. In an exemplary embodiment, the pre-determined decision threshold for a transaction to be classified as potentially fraudulent activity for a specific a model is 0.2 (while 0.5 constitutes an unbiased threshold, having no inclination towards a specific decision in a binary classification problem, such as between flagging a given transaction as legitimate or potentially fraudulent activity).

Moreover, F-beta scores can be used for evaluation of different classifiers, while favoring correct detection of fraudulent actions over flagging normal actions as potentially fraudulent. F-beta is the harmonic mean of precision and recall. Maximizing precision will minimize the occurrence of false-positive errors (i.e., missing a fraudulent action), whereas maximizing recall will minimize the false-negative errors (i.e., erroneously flagging a normal action as fraudulent). Thus, while beta=1 (F-1 score) results in weighting both precision and recall equally, beta<1 gives more weight to precision over recall, and beta>1 gives more weight to recall over precision. Commonly, when recall is favored over precision, an F-beta metric with beta=2 (F-2 score) is used as an evaluation metric.

Machine learning models enable continuous, online learning provided that new data (transactions) together with corresponding labels (“fraud” and “no fraud” class labels) are available. This enables further improvement of the performance of the machine learning classifiers as more data is collected and the machine learning classifier is retrained.

In some embodiments, new real-world data is collected through anonymized use of the disclosed system. In some embodiments, administrators at each facility using the disclosed system may provide the system with additional feedback on decisions (i.e., an indication whether a flagged transaction was actually a fraudulent activity).

Fraud Detection: Anomaly Detection (No Fraudulent Actions Included in Training)

Example 1: To examine the use of a machine learning algorithm for fraud detection, we used the isolation forest model trained and tested on a synthetic dataset generated by a simulation similar to that discussed previously for synthetic data generation. The simulation data comprises data collected over a simulation for a full year. Data includes all actions involving the administration of the drug fentanyl by anesthetists during that period (n=14,186). Two features were used: the amount of drug administered to the patient, and the amount of drug remaining in the anesthetist's syringe after administration.

Data was randomly divided into a training (75%; n=10,639) and test (25%; n=3,547) subsets, both containing normal actions only, generated in the simulation. In order to introduce fraudulent actions in the simulation data, an additional subset of outliers, imitating abnormal values indicative of the actions associated with a potentially fraudulent activity, was generated by multiplying the values of each feature of each datapoint in the test subset by weights randomly sampled from a unitary distribution in the range of [10,14]. Then, the outlier datapoints were added to the test subset (n=7,094).

Model parameters include: 200 trees (estimators); use of both two features to train each base estimator; use of 256 samples to train each base estimator; contamination ratio of 0.01. The model was trained on the training set and a decision function was fit. Then, model predictions for both test and outlier datapoints were obtained. Finally, a decision threshold for anomaly score was determined as 0.05 (i.e., scores lower than 0.05 are considered as anomalies) in order to reduce the risk of false negatives, at the price of increasing the chance of false positives (as missing potential fraudulent actions is more costly than increasing the chance of actions being flagged as potentially fraudulent).

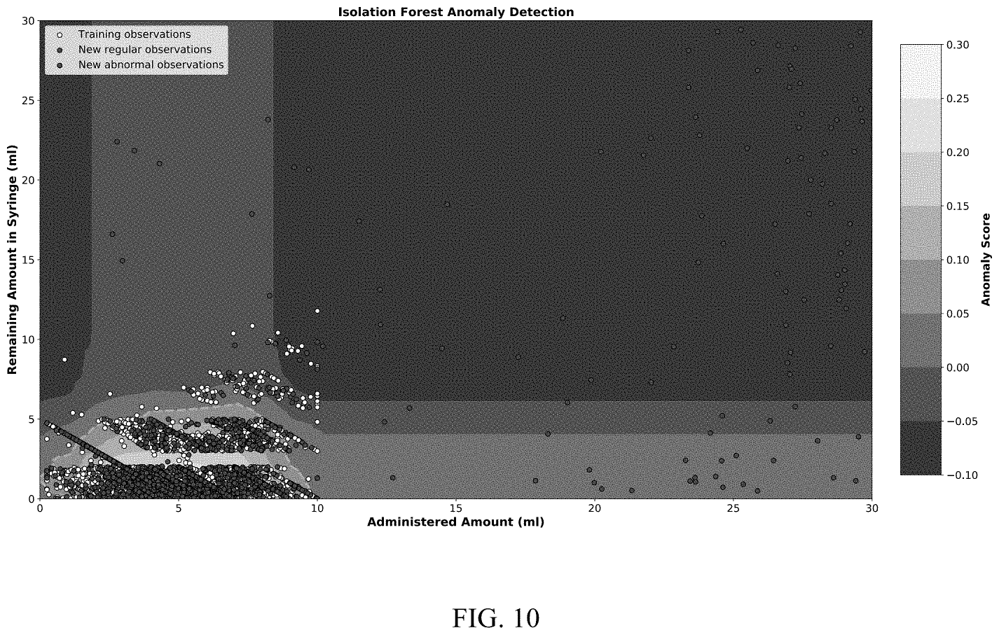

Results for anomaly detection using the isolation model are depicted in FIG. 10 and Table 3. Overall, 7,008 observations were correctly identified (3,462 and 3,546 normal and outlier datapoints, respectively), while 86 observations were mislabeled (85 normal observations labeled as outliers, and one outlier labeled as normal). Accuracy was 0.9879, with a precision score of 0.9766, a recall score of 0.9997, and an F-2 score of 0.9950. In this example, the training data was unlabeled, that is, it did not include any information about the transaction being fraudulent or not.

FIG. 10 illustrates anomaly score contour of an isolation forest model for synthetic training and test datasets generated by the simulation, and an artificial dataset of outliers. Contour color indicates decision function values (i.e., average anomaly scores across all forest trees), with darker color indicating higher anomaly scores. Decision threshold is marked by a dashed line; a conservative decision threshold of 0.05 was set.

TABLE 3
Confusion matrix for isolation forest model having a decision
threshold of 0.05.

| | No flag | Fraud flag |
|---|---|---|
| Normal Observations | 3,462 | 85 |
| Outlier Observations | 1 | 3,546 |

Fraud Detection: Action Classification (Fraudulent Actions Included in Training)

Example 2: Data for one full year was generated using the simulation similar to Example 1, with the difference being that all anesthetists had a chance of 0.01-0.05 to engage in fraud every time each of them decided to administer a drug to a patient. More specifically, each anesthetist agent was assigned with a probability randomly sampled from a uniform distribution in the range of [0.01, 0.05], indicating the chance of that agent to engage in a fraudulent action for some actions. In this example, one possible fraudulent action was made possible: increasing the amount of drug administered to a patient, and the amount of drug retrieved from the agent's drug kits for this purpose.

For cases that an agent was randomly selected to engage in a fraudulent activity, the originally chosen drug administration amount was multiplied by some number randomly sampled from a uniform distribution, in the range of [3,4]; the amount of drug to retrieve was set to this amount, multiplied by a random number from the uniform distribution, in the range of [1,3].

Generated data includes all actions involving the administration of the drug Fentanyl by anesthetists during that period (n=16,512; 16,284 normal and 228 fraud actions). Two features were used: the amount of drug administered to the patient, and the amount of drug remaining in the anesthetist's syringe after administration.

To detect outliers, a z-score was calculated for each observation as

$${z}_{ij} = \frac{{x}_{ij} - {μ}_{jk}}{{σ}_{jk}} ,$$

where xᵢⱼ is the value of feature j of observation i, labeled as belonging to class k; μⱼₖ and σⱼₖ are the mean and standard deviation of feature j for all observations of class k. Outliers were detected for each class separately (i.e., normal and fraudulent actions) and removed from analysis. Overall, 198 outliers were detected and removed from analysis: 188 normal actions (1.15% of all normal actions) and 10 fraudulent (4.39% of all fraudulent actions). Consequently, the analyzed dataset consisted of 16,314 observations (16,096 normal and 218 fraudulent actions; 1.34% fraudulent actions). Data was next pseudo-randomly divided into training (75%) and test (25%) subsets in a stratified fashion (i.e., a similar proportion of fraud actions was kept in both subsets). Finally, the training set was comprised of 12,235 observations (12,064 and 171 normal and fraudulent actions, respectively; 1.40% fraudulent actions), and the test set consisted of 4,077 observations (4,022 and 57 normal and fraudulent actions, respectively; 1.40% fraudulent actions).

Next, a grid search was conducted to obtain an optimal model. Five machine-learning classifier types were included: logistic regression, support vector machine (SVM), random forest, adaptive boosting (AdaBoost), and gradient boosting. For each classifier type, a range of values was predetermined for multiple hyperparameters. Then, a single model having each of all the possible combinations of hyperparameter values was trained and evaluated via 3-fold cross-validation on the training dataset, with the data distributed across folds in a stratified fashion (so that each fold has a similar number of fraud actions). Accuracy, precision, recall and F-2 scores were calculated for each cross-validation split, and each the values were averaged across folds. The optimal model was chosen based on having the highest F-2 score, then trained on the full training set, and finally examined on the test set. The grid search concluded with a gradient boosting model found as optimal (Tables 4 and 5). Overall, 4,061 observations were correctly identified (4,018 and 43 normal and fraudulent actions, respectively), while 18 observations were mislabeled (four normal actions labeled as fraudulent, and 14 fraudulent actions labeled as normal).

TABLE 4
Evaluation results and hyperparameters of an obtained optimal gradient
boosting model

| | Accuracy | | Precision | | Recall | | F-2 | |
|---|---|---|---|---|---|---|---|---|
| Cross-validation | Mean: | 0.997 | Mean: | 0.957 | Mean: | 0.795 | Mean: | 0.823 |
| | SD: | 0.001 | SD: | 0.004 | SD: | 0.079 | SD: | 0.069 |
| | Split 1: | 0.997 | Split 1: | 0.960 | Split 1: | 0.842 | Split 1: | 0.863 |
| | Split 2: | 0.995 | Split 2: | 0.951 | Split 2: | 0.684 | Split 2: | 0.725 |
| | Split 3: | 0.998 | Split 3: | 0.961 | Split 3: | 0.860 | Split 3: | 0.878 |

| | | | | |
|---|---|---|---|---|
| Test | 0.996 | 0.915 | 0.754 | 0.782 |

| | |
|---|---|
| Feature Importance | Administered drug amount: 0.574 |
| | Remaining drug amount in syringe: 0.426 |
| Tuned | learning_rate: 1; loss: exponential; max_depth: 8; max_features: sqrt; |
| Hyperparameters | min_impurity_decrease: 0.1; min_samples_leaf: 4; n_estimators: 2000; |
| | n_iter_no_change: 100; random_state: 99; subsample: 0.8 |

TABLE 5
Confusion matrix for the optimal model determined through a
grid search, evaluated on the test set

| | No flag | Fraud flag |
|---|---|---|
| Normal Observations | 4,018 | 4 |
| Fraud Observations | 14 | 43 |

Use of the Invention: Use of Immutable Databases

Example 3: Next, an exemplary overall workflow where the disclosed invention is used is presented. In some embodiments, the overall workflow associated with using the disclosed invention for tracking sensitive medication involves the following stages:

i) each sensitive medication pack is tagged with a small identification tag (e.g., barcode sticker such as a binary barcode or a 2-dimensional QR code, RFID tag, near field communication tag) and scanned by a user (e.g., pharmacy staff, technician, etc.) using a transaction recording module such as a smartphone application before being stored at a storage area (e.g., pharmacy vault, access-controlled area, designated shelf, or other area). Alternatively, the existing unique serial number of the sensitive medication pack is used instead of tagging sensitive medication packs with a small identification tag. Alternatively, a non-unique identification tag such as NDC is used instead of tagging sensitive medication packs with a small identification tag.

The transaction recording module performs user authentication using biometrics capabilities (e.g., fingerprint scan or face scan or extracting at least one facial feature to be used for identification of the person based on facial features) or a username and password combination. Information related to the transaction such as time, date, user, location, unique identification number (if any), NDC or other identifying information for the drug, is transmitted and recorded in an immutable database such as a blockchain.

ii) as part of transporting the sensitive medication from one location to the next (e.g., transporting from the pharmacy vault to an automated dispensing cabinet or from pharmacy to administer to patient), the person in charge of such transport scans each sensitive medication pack and records receiving the custody of such sensitive medication packs by digitally signing the transfer of sensitive medication from the storage area to a secondary location. In some embodiments, digital signing involves capturing an actual imprint of a signature drawn on a paper or surface, entering a personal identification number (PIN), biometric-based authentication methods, use of private/public key pairs for signing digital certificates, or other digital signature methods known to one skilled in the art.

If this process involves more than one user, where the first user removes the sensitive medication from the storage area and transfers custody or ownership of the sensitive medication to a second user, the transaction recording module assigned to each person is used to digitally sign the transfer of custody or ownership from the first person to the second person (e.g., each person can use their smartphone application to transfer or receive custody of the sensitive medication pack and sign the digital transfer certificate to digitally record the transfer of custody).

Transaction data comprising sender and recipient identities, time, date, and current location, and ID number (associated with the tag) of each sensitive medication pack (if available), drug type and NDC (if available), location, and patient identification tag (if any), is recorded in the immutable database;

iii) if the sensitive medication has to be moved to a secondary storage area, the person in possession of the sensitive medication scans sensitive medication packs (by scanning the tag) before placing them in the secondary storage area. In some embodiments, the secondary storage area is an automated dispensing cabinet or an access-controlled area or any designated area for a sensitive medication or a medical kit for storing sensitive medications. All transaction data including the person's identity, time, date, location, and ID number associated with the tag for all sensitive medication packs are recorded in the immutable database;

iv) before administration to the patient or wasting (discarding) the sensitive medication, the user of the system (e.g., the nurse) scans the sensitive medication pack with the transaction recording module (e.g., smartphone application) after removal from the storage area (either original storage area or secondary storage area or a medical kit). The user's identity, time, date, and location, and the ID number of the tag associated with the removed sensitive medication pack are recorded in the immutable database; and

v) in the last step, the user scans the sensitive medication pack using the smartphone app after administering the sensitive medication to the patient and/or wasting (i.e., discarding) the sensitive medication.

In some embodiments, an external camera (e.g., a wall mounted camera, a camera located in a location inside the room, and/or other configuration of cameras typically used for video monitoring) is used to record the process of storing sensitive medication in storage. Additionally, an external camera (e.g., a wall mounted camera, a camera located in a location inside the room, and/or other configuration of cameras typically used for video monitoring) may be used to record the process of removing sensitive medication packs by a certain user at a specific location or transferring the custody of the sensitive medication pack from one person to another person.

In some embodiments, if a vial or ampule or syringe or a single pill pack is used and the user takes a picture of the empty vial or ampule or syringe or pack using the image and video processing module (e.g., the smartphone app and smartphone camera) after administering the sensitive medication to the patient and/or wasting the sensitive medication. In some embodiments if a tablet or capsule or pill is used, a picture of the empty pill pack is recorded using the image and video processing module (e.g., smartphone app and smartphone camera). In some embodiments, the user or nurse records a video of the process of administering the sensitive medication to the patient orally or by injection (or by any other clinical method) and/or records the process of wasting the sensitive medication using the image and video processing module.

In some embodiments a smartphone application and smartphone camera are used to record the video of the process of administration of sensitive medication to the patient and/or the process of wasting the sensitive medication.

In some embodiments a wearable camera or a head-mounted augmented reality headset or glasses with camera are used to record the video of the process of administration of sensitive medication to the patient and/or the process of wasting the sensitive medication. In some embodiments, an external camera (e.g., a wall mounted camera, a camera located in a location inside the room, and/or other configuration of cameras typically used for video monitoring) is used to record the process of administration of the sensitive medication to the patient and/or wasting the sensitive medication. In some embodiments, a computer vision-based software system automatically confirms if the sensitive medication was administered or wasted in the video by recognizing people in the field of view and detecting an activity based on the movement of various anatomical landmarks. In some embodiments, methods described in Vrigkas, Michalis, Christophoros Nikou, and Ioannis A. Kakadiaris, “A review of human activity recognition methods,” Frontiers in Robotics and AI 2 (2015):28 are used.

In some embodiments, a computer vision-based software system automatically confirms if the vial or ampule or pack is empty in an image using deep learning and other classification methods. In some embodiments, methods described in Rawat, Waseem, and Zenghui Wang, “Deep convolutional neural networks for image classification: A comprehensive review,” Neural Computation 29:9 (2017): 2352-2449 are used. A computer vision software is used to analyze a picture and confirm if the container such as the pill pack or ampule or vial is empty. A computer vision software is used to identify at least two persons in the field of view and to identify if one person administers the medication to a second person by identifying activities associated with the administration process. A computer vision software is used to identify at least one person in the field of view and to identify if the person wastes the medication by identifying activities associated with the wasting process.

In scenarios described above where an external camera (e.g., a wall mounted camera, a camera located in a location inside the room, and/or other configuration of cameras typically used for video monitoring) is used, computer vision techniques such as neural networks (including deep neural networks) can be used to identify the identity of the subject based on facial features, wherein facial features of the person of interest is compared with known facial features of different subjects in the database to identify a match. In some embodiments, matching the faces (face identity classification) is performed by a neural network or a support vector machine classifier. In some embodiments, methods described in Trigueros, Daniel Sáez, Li Meng, and Margaret Hartnett, “Face recognition: From traditional to deep learning methods,” arXiv preprint arXiv:1811.00116 (2018) are used for facial recognition activities.

Computer vision techniques such as neural networks (including deep neural networks) can be used to track various anatomical body parts and joint positions to estimate a pose of the subject and to identify certain actions such as retrieving a drug from the storage, or filing a syringe, or administering to patient, or wasting.

In all cases above, the image of an empty vial or pack or container or the video segment showing the sensitive medication administration process and/or sensitive medication wasting process is assigned a unique identification number by a software system. This image number along with other transaction data comprising the user's identity, time, date, location, and ID number associated with the sensitive medication pack (if available), NDC number, and the patient ID number (if available) are recorded in the immutable database.

The disclosed invention is advantageous for multiple reasons. In some embodiments, the disclosed invention can reduce paperwork and increase efficiency. As part of the process, user identities are checked using the smartphone app through either a username/password or biometric-based authentication mechanisms on new smartphones such as fingerprint or face scanning. In addition, the transfer of sensitive medications from one person to another is recorded using a digital transfer certificate in the immutable database. The digital transfer certificate is a recorded transaction involving transfer data (e.g., identification of sensitive medication packs, time, date, etc.) and electronic signature defined as the identity of the sender and recipient validated by the biometric capabilities of the smartphone (e.g., face identification, finger print identification) or a username/password combination. Recording this data in the immutable database such as a blockchain ensures that the transfer information cannot be modified in the future.

In some embodiments, a computation engine that uses data (recorded in the immutable database such as the blockchain) is used to detect drug diversion or generate red flags related to drug diversion. In some embodiments, the computation engine uses data from the immutable database and data from EMRs and automated dispensing cabinets to detect drug diversion or generate red flags related to drug diversion. In some embodiments, a framework based on machine learning to detect anomalies in data (i.e., drug diversion) can be used. The first step of the process involves feature extraction from the data recorded in the immutable database, where a series of metrics are extracted from the raw data. Features comprise the number of sensitive medication packs in storage; the number of vials removed by each person at any given time; the time between a specific sensitive medication pack removal from the storage and storing, using, or wasting that same sensitive medication pack; correlation between administration of the sensitive medication to a patient and reported pain scores stored on the EMR; correlation between administration of the sensitive medication to a patient and patient's disease status stored on the EMR; among others.

In some embodiments, the machine learning framework to detect drug diversion is an autoencoder neural network (feedforward non-recurrent) apparatus to detect anomalies. An autoencoder is a neural network apparatus (with an input layer, one or more hidden layers, and an output layer) and it is capable of encoding data (i.e., performing dimensionality reduction) in an unsupervised manner. Autoencoders can be used to find “hidden patterns” in data automatically. Here, by setting the number of inputs to equal the number of outputs, the autoencoder tries to reconstruct an input by first mapping the data into a lower dimensional space and remapping it back to the same dimensions. If the neural network is trained on data with no anomaly, the weights of the autoencoder will converge to values where extracted features can be accurately reconstructed. Next, if it is presented with data where there is an underlying anomaly, the reconstruction will involve an error, as the pre-trained autoencoder is not “used to” the new data patterns (i.e., has not seen this specific pattern before). This technique can be used to detect anomalies if the reconstruction error is higher than a specific threshold.

A GUI is used to display the current state of the sensitive medication packs. The state comprises the total number of sensitive medication packs in the inventory, last scanned time, date for each sensitive medication pack, and the identity of the person last interacting with each sensitive medication pack. The GUI is also used to communicate red flags associated with a potential drug diversion to the user.

A notification module is used to send alerts and notification to a supervisor alerting them to a detection of a potential drug diversion.

The components of the system include the immutable database which receives transaction data from the transaction recording module, and image and video from the image and video processing module. The transaction data and image and/or video can be sent to the computation engine for processing and the results of the process can be displayed on the computation engine GUI to review existing data. If the computation engine detects a potential drug diversion (or a red flag) it sends a notification the appropriate person. This notification system is referred to as the notification module.

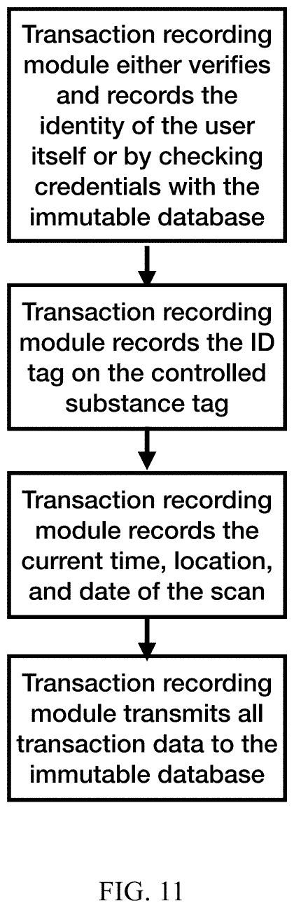

FIG. 11 illustrates the steps that the transaction recording module takes as part of recording all transaction data and communicating the data with the immutable database. The process involves verification of the user by either authenticating the user or asking the immutable database for authentication. It then records the unique ID tag (if available) associated with a scanned sensitive medication pack by the user. The transaction recording module records current time, date, and location of the scan, and next, transmits all data to the immutable database.

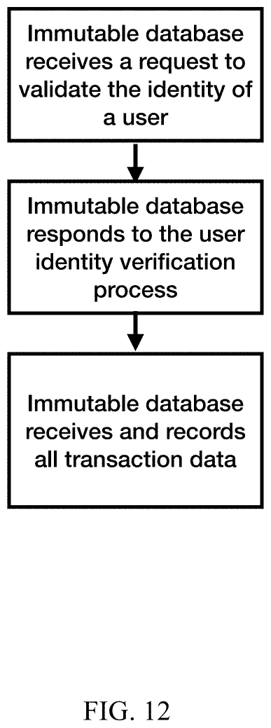

FIG. 12 illustrates the steps that the immutable database takes as part of recording all transaction data and receiving such data from at least one transaction recording module. If the transaction recording module determines, it may send a request to the immutable database to verify the identity of the user by either checking a username and password combination or comparing a submitted biometrics record against existing record of authorized users in the immutable database. The immutable database then performs the user verification and transmits the decision of such verification to the transaction recording module. Depending on the decision (verified or not verified) the transaction recording module may determine to send all transaction data to the immutable database for recordation.

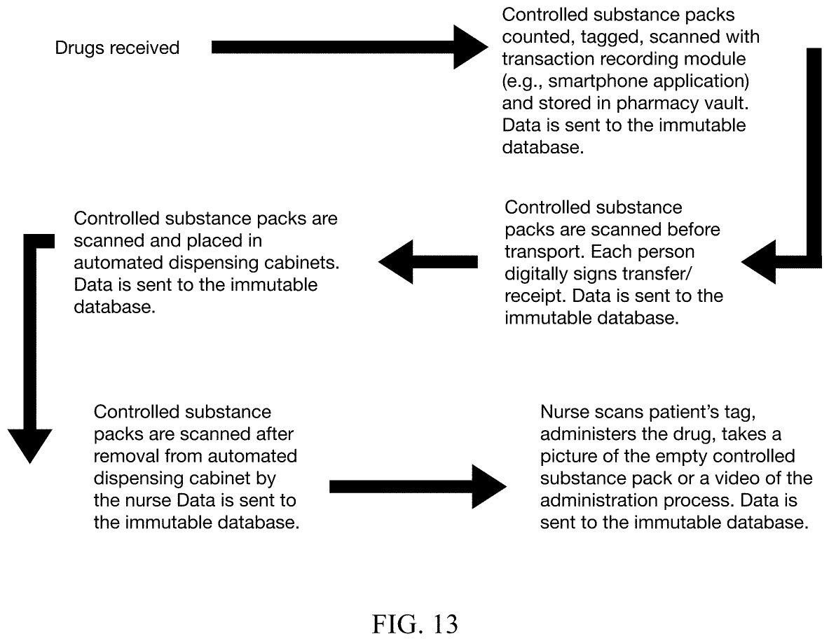

FIG. 13 illustrates an embodiment of the overall workflow associated with using the disclosed invention for tracking sensitive medication in a hospital or healthcare facility. The sensitive medication is first received. Next, sensitive medication packs are counted, tagged, scanned with the transaction recording module (e.g., smartphone application) and stored in the pharmacy vault. Data is sent to the immutable database for recording. Next, sensitive medication packs are scanned before transport. Each person digitally signs for transfer or receipt of each sensitive medication pack. Data is sent to the immutable database.

Next, sensitive medication packs are scanned and placed in automated dispensing cabinets. Data is sent to the immutable database. Sensitive medication packs are scanned after removal from automated dispensing cabinet by the nurse. Data is sent to the immutable database. Finally, the nurse or other administering party scans the patient's tag, scans the sensitive medication pack, administers the drug, takes a picture of the empty sensitive medication pack, or a video of the administration process. Data is sent to the immutable database.

Immutable Databases

While traditional databases such as relational databases may be used to record required transactions associated with the system, immutable databases may also be employed to ensure an auditable trail for all transaction exists.

In some embodiments, the Hyperledger Fabric, an open-source blockchain framework geared towards enterprise applications which was founded by the Linux Foundation, can be used. Hyperledger Fabric is a permissioned (i.e., private) blockchain to control access to the sensitive data (e.g., healthcare and patient-related data) that will be recorded on the blockchain. In a permissioned apparatus, a main identity provider manages and controls access to the network. An advantage of the Hyperledger Fabric is its flexibility in implementation including pluggable consensus protocols and general-purpose programming languages. In some embodiments, the practical Byzantine fault-tolerant consensus protocol is used, where large computational power requirement by other protocols is avoided making it more attractive for adoption by hospitals and healthcare facilities. In some other embodiments, the proof of work protocol used by bitcoin is used.

In some embodiments, smart contracts for blockchains are used to record transactions. In this embodiment, transactions discussed above including transportation of sensitive medication packs from one storage area to another, signing digital certificates upon delivery or exchange of sensitive medication packs between users, adding and removing items from inventory, receiving of new inventory by the supplier to be added to the inventory are implemented through smart contracts. In some embodiments, methods described in Rouhani, Sara, and Ralph Deters, “Security, performance, and applications of smart contracts: A systematic survey,” IEEE Access 7 (2019): 50759-50779 can be used to implement such smart contracts.

In some embodiments, the Bitcoin (peer-to-peer) protocol is used to record transactions. In some embodiments, the Ethereum protocol is used to record transactions and the related smart contracts.

In some embodiments, the multichain framework is used to enhance interoperability between different blockchain types and integration with smart contracts. Exemplary embodiments include Brand Protocol, Chainlink, and Polkadot.

In some embodiments, permission frameworks discussed in Polge, Julien, Jeremy Robert, and Yves Le Traon, “Permissioned blockchain frameworks in the industry: A comparison,” Ict Express 7.2 (2021): 229-233 are used to implement the immutable database. In some embodiments, blockchain consensus protocols discussed in Cachin, Christian, and Marko Vukolić, “Blockchain consensus protocols in the wild” arXiv preprint arXiv:1707.01873 (2017) are used. In some embodiments, an immutable database is implemented using the methods discussed in Kuo, Tsung-Ting, Hugo Zavaleta Rojas, and Lucila Ohno-Machado, “Comparison of blockchain platforms: a systematic review and healthcare examples,” Journal of the American Medical Informatics Association 26.5 (2019): 462-478.

Use of the Invention: Asset Tracking during Transportation and Authentication of Packages

Example 4: Next, an exemplary overall workflow where the disclosed invention is used within a supply chain framework is presented. In some embodiments, the overall workflow associated with using the disclosed invention for tracking sensitive medication involves the following stages:

i) Sensitive medication packs are assembled into packages of certain size. In some embodiments, a package includes 1, 5, 10, 20, 25, 30, 40, 50, 75, 100, 200, 250, 500 or more vials or ampules or syringes by the manufacturer. The manufacturer attaches an identification tag to each package comprising a unique serial number (or tracking number), lot number, expiration date, and NDC. Packages are scanned by a transaction recording module upon shipment to a distributor. At least one image and/or video is recorded using an image and video processing module. The image and/or video serves as the original point of reference to compare subsequent images and/or videos captured throughout the transportation and exchange process. A transaction is recorded to record the creation of this package. Transaction data comprises unique ID, expiration date, lot number, expiration date, time and date, location, and the user recording the transaction.

ii) The distributor receives packages from the manufacturer and scans the identification tag using a transaction recording module to confirm receipt of shipment. A transaction is recorded to record this receipt. Transaction data comprises unique ID, expiration date, lot number, expiration date, time and date, location, and the user receiving the package and the user providing (handing in) the package. At least one image and/or video is recorded using an image and video processing module. The image and/or video serves as proof that such package was actually in the position of the distributor when the identification tag was scanned (and a fake identification tag was not scanned).

iii) The distributor ships packages to another distributor or to a healthcare facility. The receiver (healthcare facility or a second distributor) scans the identification tag using a transaction recording module to confirm receipt of shipment. A transaction is recorded to record this exchange. Transaction data comprises unique ID, expiration date, lot number, expiration date, time and date, location, and the user receiving the package and the user providing (handing in) the package. At least one image and/or video is recorded using an image and video processing module. The image and/or video serves as proof that such package was actually in the position of the distributor when the identification tag was scanned (and a fake identification tag was not scanned).

iv) Transactions are recorded by the database module (which may use a traditional database or immutable database to record transactions). If an immutable database is used, smart contracts can be used to certify exchange of packages and trigger fulfillment of contractual obligations when packages have been exchanged. The database module can also communicate with a secondary database responsible for recording packages shipped by the manufacturer. In some embodiments, the database module confirms the unique serial number scanned by a transaction recording module is authentic by cross-referencing the same serial number in a secondary database. This ensures that this package does not contain counterfeit medications.

In some embodiment, image and/or video recorded by the manufacturer, and one or more distributor, and/or receiver and recorded by one or more image and video processing modules are available for audit to ensure that the package is the same and has not been altered or changed or swapped during the transportation process. In some embodiments, a computer vision algorithm is used to compute the similarity between the images and the confirm that they are the one and the same package.

The computation engine module is responsible to detect any anomalies. The transaction analysis subcomponent reviews transaction data recorded by the manufacturer, distributor(s), and the receiver to identify potentially fraudulent activities. The classification approaches presented earlier in this invention can be used to identify such potentially fraudulent activities and communicate the results with the user with appropriate role through the computation engine graphical user interface.

In some embodiments, the manufacturer, the distributer, and the receiver use their own specific type of immutable database. In some embodiments, a multichain framework is used to communicate across multiple types of immutable databases used by various system participants. In some embodiments, permission frameworks discussed in Polge, Julien, Jeremy Robert, and Yves Le Traon, “Permissioned blockchain frameworks in the industry: A comparison” Ict Express 7.2 (2021): 229-233 are used to implement the immutable database. In some embodiments, blockchain consensus protocols discussed in Cachin, Christian, and Marko Vukolie, “Blockchain consensus protocols in the wild” arXiv preprint arXiv:1707.01873 (2017) are used. In some embodiments, the immutable database is implemented using the methods discussed in Kuo, Tsung-Ting, Hugo Zavaleta Rojas, and Lucila Ohno-Machado,” Comparison of blockchain platforms: a systematic review and healthcare examples” Journal of the American Medical Informatics Association 26.5 (2019): 462-478.

It is intended that one of skill in the art will take the examples, tables, and information described herein as examples of the system, apparatus, and method of the invention; they are not intended to limit the invention to these specific embodiments.

1. A system to track sensitive medication packs, comprising:
a. a database module configured to receive transaction data from at least one transaction recording module and verify a user's identity using a biometric verification process or a username and password combination, in combination with a previously-recorded verification data stored in the database module, wherein said biometric verification process or a username and password combination is received from the transaction recording module;
b. an image and video processing module in operable communication with the database module and at least one transaction recording module, wherein the image and video processing module comprise
i. an image and video capture subcomponent for recording image and video data, and
ii. an image and video processing subcomponent for extracting drug information, wherein said extracted drug information comprises at least one barcode on a sensitive medication pack;
c. the at least one transaction recording module, which is in operable communication with the image and video processing module for receiving the extracted drug information and wherein the at least one transaction recording module is also in operable communication with the database module, wherein the at least one transaction recording module is configured to
i. verify a user's identity using a biometric verification process or a username and password combination, in combination with a previously-recorded verification data stored in the database module;
ii. generate transaction data comprising at least the drug information and the user's identity verification response;
iii. transmit transaction data to the database module; and
iv. receive a confirmation from the database module that transaction data was successfully recorded; and
d. a computation engine module, which receives the transaction data from the database module, wherein the computation engine module comprises
i. a transaction analysis subcomponent for detecting a potential fraudulent activity based at least on the transmitted transaction data, and
ii. a computation engine graphical user interface subcomponent to communicate the detection of the potentially fraudulent activity to a user.

2. The system of claim 1, wherein the image and video processing module assigns a unique identification number to a recorded image of the sensitive medication packs.

3. The system of claim 1, wherein the image and video processing module assigns a unique identification number to a recorded video of the sensitive medication administration process.

4. The system of claim 3, wherein the recorded video and the unique identification number are transmitted to the database module.

5. The system of claim 1, wherein the computation engine module sends a notification to the user when the potentially fraudulent activity is detected.

6. The system of claim 5, wherein said notification is sent to the user via SMS.

7. The system of claim 1, wherein the transaction analysis subcomponent uses a machine learning classifier.

8. The system of claim 1, wherein the transaction analysis subcomponent uses a static rule-based classifier.

9. The system of claim 1, wherein sensitive medication packs are controlled substance packs.

10. A system for waste of sensitive medication packs, comprising:
a. a database module configured to
i. receive witness user login data from a witness user transaction recording module, verify said received witness user login data, and generate a witness user access token for the witness user transaction recording module if such verification is successful, wherein verification of the witness user login data is done using a biometric verification process or a username and password combination, in combination with a previously-recorded verification data stored in the database module;
ii. receive waster user login data from a waster user transaction recording module, verify said received waster user login data, and generate a waster user access token for the waster user transaction recording module if such verification is successful, wherein verification of the waster user login data is done using a biometric verification process or a username and password combination, in combination with a previously-recorded verification data stored in the database module;
b. the witness user transaction recording module, wherein the witness user transaction recording module is in operable communication with at least one image and video processing modules for receiving a witness waste request code and wherein witness user transaction recording module is also in operable communication with the database module, wherein the witness user transaction recording module is configured to
i. generate a witness waste confirmation code,
ii. generate witness transaction data comprising at least the witness waste request code and the waster user access token,
iii. transmit witness transaction data to the database module;
c. the waster user transaction recording module, wherein the waster user transaction recording module is in operable communication with the at least one image and video processing modules for receiving a witness waste confirmation code and wherein waster user transaction recording module is also in operable communication with the database module, wherein the waster user transaction recording module is configured to
i. generate a witness waste request code,
ii. generate waster transaction data comprising at least the witness waste confirmation code and the waster user access token,
iii. transmit waster transaction data to the database module; and
d. the image and video processing module in operable communication with the database module, the waster user transaction recording module, and the witness user transaction recording module, wherein the image and video processing module comprises
i. an image and video capture subcomponent for recording image and video data, and
ii. an image and video processing subcomponent for
a. extracting the witness waste request code, wherein said extracted witness waste request code comprises at least one barcode displayed on the waster user transaction recording module graphical user interface,
b. extracting the witness waste confirmation code, wherein said extracted witness waste confirmation code comprises at least one barcode displayed on the witness user transaction recording module graphical user interface.

11. The system of claim 10, further comprising a computation engine module, which receives the witness transaction data and waster transaction data from the database module, wherein the computation engine module comprises
a. a transaction analysis subcomponent for detecting a potential fraudulent activity based at least on the witness transaction data and waster transaction data; and
b. a computation engine graphical user interface subcomponent to communicate the detection of the potentially fraudulent activity to a user.

12. The system of claim 11, wherein the computation engine module sends a notification to the user when the potentially fraudulent activity is detected.

13. The system of claim 11, wherein the transaction analysis subcomponent uses a machine learning classifier.

14. The system of claim 11, wherein the transaction analysis subcomponent uses a static rule-based classifier.

15. The system of claim 11, wherein sensitive medication packs are controlled substance packs.
